(19) United States
(12) Patent Application Publication
(10) Pub. No.: US 2011/0200543 A1
(43) Pub. Date: Aug. 18, 2011

(54) ANHYDROUS FLUID FILTERING COMPOSITION COMPRISING AN OILY PHASE, A PARTICULAR TRIAZINE FILTER AND AN OIL THICKENING OR GELLING RHEOLOGICAL AGENT

(75) Inventor: Martin Josso, Paris (FR)

(73) Assignee: L'OREAL, Paris (FR)

(21) Appl. No.: 12/966,373

(22) Filed: Dec. 13, 2010

Related U.S. Application Data
(60) Provisional application No. 61/286,644, filed on Dec. 15, 2009.

(30) Foreign Application Priority Data
Dec. 11, 2009 (FR) 0958876

(51) Int. Cl. A61K 8/92; A61Q 17/04

(52) U.S. Cl. 424/60; 424/59

(57) ABSTRACT

The present invention relates to an anhydrous fluid composition comprising, in a cosmetically acceptable medium:
a) at least one oily phase and
b) at least one particular triazine UV filter of formula (I) and
c) at least one rheological agent for thickening or gelling the oily phase.

The present invention also relates to the use of said composition for the manufacture of products for the cosmetic treatment of the skin, nails, hair, eyelashes, eyebrows and/or scalp, notably care products, sun protection products, make-up products.

The present invention relates to an anhydrous fluid composition comprising, in a cosmetically acceptable medium:

a) at least one oily phase and
b) at least one particular triazine UV filter of formula (I) and
c) at least one rheological agent for thickening or gelling the oily phase.

It is known that light of wavelength between 280 nm and 400 nm permits tanning of the human epidermis, and that radiation of wavelength more particularly between 280 and 320 nm, known under the name UV-B, causes erythema and skin burns which can adversely affect the development of a natural tan. For these reasons, as well as for aesthetic reasons, there is a constant demand for means for controlling natural tanning so as to control the colour of the skin; it is therefore necessary to filter this UV-B radiation.

It is also known that UV-A radiation, with wavelengths between 320 and 400 nm, which causes tanning of the skin, can induce a deterioration of the latter, notably in the case of sensitive skin or skin that is constantly exposed to sunlight. In particular, UV-A radiation causes loss of elasticity of the skin and the appearance of wrinkles, leading to premature skin ageing. It promotes triggering of the erythematous reaction or amplifies this reaction in certain subjects and can even be the cause of phototoxic or photoallergic reactions. Thus, for aesthetic and cosmetic reasons such as maintaining the natural elasticity of the skin for example, more and more people want to control the effect of UV-A radiation on their skin. It is therefore desirable to filter UV-A radiation as well.

To provide protection of the skin and keratinous materials against UV radiation, sunscreen compositions are generally used, comprising organic filters, active in the UV-A range and active in the UV-B range.

Numerous cosmetic compositions intended for photoprotection (UV-A and/or UV-B) of the skin have been proposed up to now. Quite particularly, there is a search for fluid formulations, which provide users with easy application on the skin.

Among the fluid sunscreen compositions proposed to date, anhydrous formulations of the sunscreen oil type are in particular demand because their application on the skin is easy and pleasant and they have good water resistance. However, they have not found wide acceptance in the market for sunscreen products because it is difficult to obtain a sun protection factor above 10 or even above 15 as well as a UVA protection factor complying with the ratio required by the various regulations relating to sunscreen products, notably above 5.

The sun protection factor (SPF) is expressed mathematically as the ratio of the irradiation time required to reach the erythema threshold with the UV filter to the time required to reach the erythema threshold without a UV filter. It is evaluated in vivo notably by the International Method published by Colipa/CTFA SA/JCIA (May 2006).

For characterizing protection against UV-A, the PPD (Persistent Pigment Darkening) method, which measures the colour of the skin observed 2 to 4 hours after exposure of the skin to UV-A, is particularly recommended and used. This method has been in use since 1996 by the Japanese Cosmetic Industry Association (JCIA) as the official test procedure for the UV-A labelling of products and is frequently used by testing laboratories in Europe and in the United States (Japan Cosmetic Industry Association Technical Bulletin. Measurement Standards for UVA protection efficacy. Issued Nov. 21, 1995 and effective of Jan. 1, 1996).

The UVA_{PPD} protection factor (UVA_{PPD} PF) is expressed mathematically as the ratio of the dose of UV-A radiation required to reach the pigmentation threshold with the UV filter (MPPDp) to the dose of UV-A radiation required to reach the pigmentation threshold without UV filter (MPPDnp).

$$U V {A}_{PPD} P F = \frac{M P P Dp}{M P P Dnp}$$

In fact, one of the main difficulties encountered in the manufacture of sunscreen oils is that, in contrast to other types of sunscreen formulations, an increase in the proportion of sun filter is not sufficient to increase the protection factor.

Thus, sunscreen oils comprising, as lipophilic organic filter, 2-[(p-(tert-butylamido)anilino]-4,6-bis-[(p-(2′-ethylhexyl-1′-oxycarbonyl)anilino]-1,3,5-triazine or “Diethylhexyl Butamido Triazone” sold under the trade name “UVASORB HEB” by SIGMA 3V are known from the prior art in patent applications WO9906014, EP845261, EP845260, EP821938, EP821940, EP800813, EP1300137, EP1027883. These sunscreen oils have a limited SPF and increasing the proportion of filter is not sufficient to increase it significantly.

Applications EP1813266 and EP2014277 have already proposed using lipophilic polyamide polycondensates, in particular an ester-terminated poly(ester-amide) polymer (ETPEA) or a polyamide polymer terminated with a tertiary amide (ATPA), in aqueous fluid sunscreen compositions of the emulsion type, in order to obtain high sun protection factors.

However, addition of such a polymer to a fluid sunscreen oil containing a large amount of sun filters can lead to an increase in viscosity of the formulation, after manufacture and over time and notably during storage at low temperature, resulting in poor flow of the composition and therefore making it difficult to use.

There is therefore a need to find new anhydrous fluid sunscreen compositions that are stable over time and give higher sun protection factors without the drawbacks mentioned above.

Now, the applicant discovered, surprisingly, that this objective could be achieved with an anhydrous fluid composition comprising, in a cosmetically acceptable medium:

a) at least one oily phase and
b) at least one particular triazine UV filter of formula (I), which will be defined in detail hereunder, and
c) at least one rheological agent for thickening or gelling the oily phase.

This discovery forms the basis of the present invention.

The present invention relates to an anhydrous fluid composition comprising, in a cosmetically acceptable medium:

a) at least one oily phase and
b) at least one particular triazine UV filter of formula (I) and
c) at least one rheological agent for thickening or gelling the oily phase.

Other characteristics, aspects and advantages of the invention will become clear on reading the detailed description presented below.

“Cosmetically acceptable” means compatible with the skin and/or its appendages, having a pleasant colour, odour and feel and not causing unacceptable discomfort (tingling, tightness, redness), that might put the consumer off using this composition.

“Fluid composition” means, in the sense of the invention, a composition that is not in solid form and whose viscosity measured with a Rheomat 180 viscosimeter at 25° C. at a rotary speed of 200 RPM after 30 seconds of rotation is less than 0.5 Pa·s and more preferably less than 0.2 Pa·s and more particularly in the range from 0.0001 Pa·s to 0.1 Pa·s.

“Anhydrous composition” means a composition containing less than 1 wt. % water, or even less than 0.5% water, and notably water-free, the water not being added during preparation of the composition, but corresponding to residual water supplied by the mixed ingredients.

“Liquid oily phase” means, in the sense of the present application, an oily phase that is liquid at room temperature (25° C.) and atmospheric pressure (760 mmHg), composed of one or more fats that are liquid at room temperature, also called oils, which are mutually compatible.

“Lipophilic” means any cosmetic or dermatological compound that can be dissolved completely in the molecular state in a liquid oily phase or can be solubilized in colloidal form (for example in the form of micelles) in a liquid oily phase.

“Oil” means a fat that is liquid at room temperature (20 to 25° C.).

“Hydrocarbon oil” means any oil comprising mainly carbon atoms and hydrogen atoms, and optionally ester, ether, fluorinated, carboxylic acid, alcohol, silicone, amine, phenyl and/or amino acid groups.

“Rheological agent for thickening or gelling the oily phase” means a compound that is able to increase the viscosity of the fatty phase of the composition or to modify its rheological properties while leaving it in liquid form.

### Triazine UV Filters of Formula (I)

The derivatives of 1,3,5-triazine according to the invention correspond to the following formula (I):

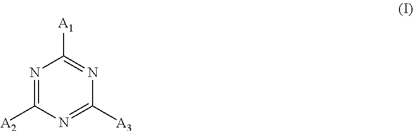

in which the radicals A₁, A₂ and A₃, which may be identical or different, denote a group with the following formula (II):

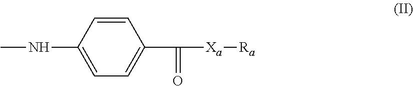

one or two of the groups Xₐ—Rₐ represent the radical —NH—Rₐ with Rₐ selected from: a linear or branched C₁-C₁₈ alkyl radical; a C₅-C₁₂ cycloalkyl radical optionally substituted with one or more C₁-C₄ alkyl radicals; a radical of the following formula (α), (β) or (ε):

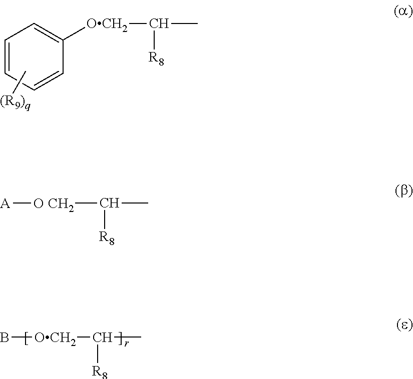

in which:
R₈ is hydrogen or a C₁-C₄ alkyl radical, preferably methyl;
R₉ is C₁-C₄ alkyl and preferably methyl;
q is an integer from 0 to 3;
r is an integer from 1 to 10;
A is a C₄-C₈ alkyl radical or a C₅-C₈ cycloalkyl radical;
B is a C₁-C₄ alkyl radical;
the other two Xₐ—Rₐ being the radical —O—Rₐ with Rₐ, which may be identical or different, selected from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (α), (β) or (ε) as defined above.

A 1,3,5-triazine that is particularly preferred from this second family is 2-[(p-(tert-butylamido)anilino]-4,6-bis-[(p-(2′-ethylhexyl-1′-oxycarbonyl)anilino]-1,3,5-triazine or “Diethylhexyl Butamido Triazone” sold under the trade name “UVASORB HEB” by SIGMA 3V and corresponding to the following formula:

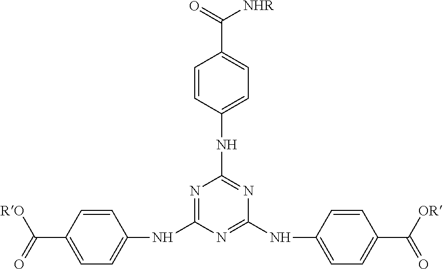

in which R′ denotes an ethyl-2-hexyl radical and R denotes a tert-butyl radical.

The triazine compound of formula (I) is preferably present in the compositions according to the invention in proportions in the range from 0.1 to 10 wt. % relative to the total weight of the composition, and preferably in the range from 1 to 6 wt. % relative to the total weight of the composition.

### Rheological Agent Thickening or Gelling the Fatty Phase

The rheological agent for thickening or gelling the oily phase is preferably present in amounts in the range from 0.1% to 10%, more preferably from 1% to 7% and even more preferably from 4% to 7% relative to the total weight of the composition.

The rheological agent for thickening or gelling the oily phase can be selected from:
(1) crystalline polymers, preferably selected from semicrystalline polymers,
(2) esters of dextrin and a fatty acid,
(3) modified hydrophobic polysaccharides,
(4) crystalline olefin copolymers
(5) crystalline polycondensates;
(6) lipophilic mineral structure-forming agents, such as lipophilic clays and hydrophobic silicas, such as pyrogenic silica with hydrophobic treatment,
(7) lipophilic polyamide polymers,
(8) lipophilic polyureas and polyurethanes,
(9) silicone polymers comprising if necessary at least one hydrocarbon unit having two groups capable of establishing hydrogen interactions selected from the ester, amide, sulphonamide, carbamate, thiocarbamate, urea, urethane, thiourea, oxamido, guanidino, biguanidino groups and combinations thereof, preferably amide groups,
(10) organic gelling agents;
(11) block copolymers;
(12) silicone elastomers;
(13) cholesteric liquid crystal agents;
(14) waxes;
   and mixtures thereof.

(1) Crystalline Polymers

### 1.1 Semi-Crystalline Polymers

“Semi-crystalline polymer” means polymers having a crystallizable moiety, crystallizable pendant and/or terminal chain or crystallizable block in the backbone and/or at the ends, and an amorphous moiety in the backbone and having a temperature of first-order reversible phase change, in particular of melting (solid-liquid transition). When the crystallizable moiety is in the form of a crystallizable block of the polymer backbone, the amorphous moiety of the polymer is in the form of an amorphous block; the semicrystalline polymer is in this case a block copolymer for example of the diblock, triblock or multiblock type, having at least one crystallizable block and at least one amorphous block. “Block” generally means at least 5 identical repeating units. The crystallizable block or blocks are then of a different chemical nature from the amorphous block or blocks.

The semicrystalline polymer according to the invention has a melting point greater than or equal to 30° C. (notably in the range from 30° C. to 80° C.), preferably in the range from 30° C. to 60° C. This melting point is a temperature of first-order change of state.

This melting point can be measured by any known method and in particular by means of a differential scanning calorimeter (DSC).

Advantageously, the semicrystalline polymer or polymers according to the invention have a number-average molecular weight greater than or equal to 1000.

Advantageously, the semicrystalline polymer or polymers of the composition of the invention have a number-average molecular weight Mn in the range from 2000 to 800 000, preferably from 3000 to 500 000, more preferably from 4000 to 150 000, notably less than 100 000, and most preferably from 4000 to 99 000. Preferably, they have a number-average molecular weight greater than 5600, for example in the range from 5700 to 99 000.

“Crystallizable chain or block” means, in the sense of the invention, a chain or block which, if alone, would pass from the amorphous state to the crystalline state, reversibly, according to whether it is above or below the melting point. A chain in the sense of the invention is a group of atoms, pendant or lateral relative to the backbone of the polymer. A block is a group of atoms belonging to the backbone, said group constituting one of the repeating units of the polymer. Advantageously, the “crystallizable pendant chain” can be a chain having at least 6 carbon atoms.

Preferably, the crystallizable blocks or chains of the semicrystalline polymers represent at least 30% of the total weight of each polymer and preferably at least 40%. The semicrystalline polymers of the invention with crystallizable blocks are block or multiblock copolymers. They can be obtained by polymerization of a monomer with reactive (or ethylenic) double bonds or by polycondensation. When the polymers of the invention are polymers with crystallizable side chains, the latter are advantageously of random form.

Preferably, the semicrystalline polymers of the invention are of synthetic origin. Moreover, they do not have a polysaccharide backbone. Generally, the crystallizable units (chains or blocks) of the semicrystalline polymers according to the invention are derived from monomer(s) with crystallizable block(s) or chain(s), used for the manufacture of the semicrystalline polymers.

According to the invention, the semicrystalline polymer can be selected from block copolymers having at least one crystallizable block and at least one amorphous block, homopolymers and copolymers bearing at least one crystallizable side chain per repeating unit, and mixtures thereof.

The semicrystalline polymers usable in the invention are in particular:
block copolymers of polyolefins with controlled crystallization, notably those whose monomers are described in EP-A-0 951 897.
polycondensates and notably of the polyester, aliphatic or aromatic or aliphatic/aromatic copolyester type,
homo- or co-polymers bearing at least one crystallizable side chain and homo- or co-polymers bearing at least one crystallizable block in the backbone, such as those described in document U.S. Pat. No. 5,156,911,
homo- or co-polymers bearing at least one crystallizable side chain in particular with fluorinated group(s) as described in document WO-A-01/19333,
and mixtures thereof.

In these two last cases, the crystallizable side chain(s) or block(s) are hydrophobic.

a) Semi-Crystalline Polymers with Crystallizable Side Chains:

We may mention in particular those defined in document U.S. Pat. No. 5,156,911 and WO-A-01/19333. They are homopolymers or copolymers having from 50 to 100 wt. % of units resulting from the polymerization of one or more monomers bearing a hydrophobic crystallizable side chain.

These homo- or co-polymers are of any nature provided that they meet the conditions stated above.

They can result from:
the polymerization, notably radical polymerization, of one or more monomers with reactive or ethylenic double bond(s) with respect to polymerization, namely with vinylic, (meth)acrylic or allylic groups.
the polycondensation of one or more monomers bearing co-reactive groups (carboxylic or sulphonic acid, alcohol, amine or isocyanate), for example polyesters, polyurethanes, polyethers, polyureas, polyamides.

Generally, these polymers are notably selected from the homopolymers and copolymers resulting from the polymerization of at least one monomer with crystallizable chain(s) which can be represented by formula (I):

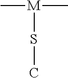

with M representing an atom of the polymer backbone, S representing a spacer, C representing a crystallizable group.

The crystallizable “—S—C” chains can be aliphatic or aromatic, optionally fluorinated or perfluorinated. “S” notably represents a group (CH₂)ₙ or (CH₂CH₂O)ₙ or (CH₂O), linear or branched or cyclic, n being an integer in the range from 0 to 22. “S” is preferably a linear group. Preferably, “S” and “C” are different.

When the crystallizable “—S—C” chains are aliphatic hydrocarbon chains, they have alkyl hydrocarbon chains with at least 11 carbon atoms and at most 40 carbon atoms and preferably at most 24 carbon atoms. They are notably aliphatic chains or alkyl chains possessing at least 12 carbon atoms and preferably they are C₁₄-C₂₄ alkyl chains. When they are fluorinated or perfluorinated alkyl chains, they have at least 6 fluorinated carbon atoms and notably at least 11 carbon atoms of which at least 6 carbon atoms are fluorinated.

As examples of semicrystalline polymers or copolymers with crystallizable chain(s), we may mention those resulting from the polymerization of one or more of the following monomers: saturated alkyl (meth)acrylates with a C₁₄-C₂₄ alkyl group, perfluoroalkyl (meth)acrylates with a C₁₁-C₁₅ perfluoro alkyl group, N-alkyl (meth)acrylamides with a C₁₄ to C₂₄ alkyl group with or without a fluorine atom, vinyl esters with alkyl or perfluoro (alkyl) chains with a C₂₄ to C₂₄ alkyl group (with at least 6 fluorine atoms per perfluoro alkyl chain), vinyl ethers with alkyl or perfluoro (alkyl) chains with a C₂₄ to C₂₄ alkyl group and at least 6 fluorine atoms per perfluoro alkyl chain, C₂₄ to C₂₄ alpha-olefins for example octadecene, para-alkyl styrenes with an alkyl group having from 12 to 24 carbon atoms, and mixtures thereof.

When the polymers result from a polycondensation, the crystallizable hydrocarbon and/or fluorinated chains as defined above are carried by a monomer which can be a diacid, a diol, a diamine, a di-isocyanate.

When the polymers according to the invention are copolymers, they contain, in addition, from 0 to 50% of groups Y or Z resulting from the copolymerization:

α) of Y which is a polar or nonpolar monomer or a mixture of the two:
When Y is a polar monomer, it is either a monomer bearing polyalkoxylated groups (notably ethoxylated and/or propoxylated), a hydroxyalkyl (meth)acrylate such as hydroxyethyl acrylate, (meth)acrylamide, an N-alkyl(meth)acrylamide, an N,N-dialkyl(meth)acrylamide for example N,N-diisopropylacrylamide or N-vinyl-pyrrolidone (NVP), N-vinyl caprolactam, a monomer bearing at least one carboxylic acid group such as (meth)acrylic, crotonic, itaconic, maleic, fumaric acids or bearing a carboxylic acid anhydride group such as maleic anhydride, and mixtures thereof.
When Y is a nonpolar monomer it can be an ester of the alkyl (meth)acrylate type, linear, branched or cyclic, a vinyl ester, an alkyl vinyl ether, an alpha-olefin, styrene or styrene substituted with a C₁ to C₁₀ alkyl group, such as α-methylstyrene, a macromonomer of the polyorganosiloxane type with a vinylic unsaturation.

“Alkyl” means, in the sense of the invention, a saturated group notably of C₈ to C₂₄, unless expressly mentioned, and preferably C₂₄ to C₂₄.

β) of Z which is a polar monomer or a mixture of polar monomers. In this case, Z has the same definition as the “polar Y” defined above.

Preferably, the semicrystalline polymers with crystallizable side chain are homopolymers of alkyl(meth)acrylate or of alkyl(meth)acrylamide with an alkyl group as defined above, and notably of C₂₄-C₂₄, copolymers of these monomers with a hydrophilic monomer preferably of a nature different from (meth)acrylic acid such as N-vinylpyrrolidone or hydroxyethyl (meth)acrylate and mixtures thereof.

### b) Polymers Bearing at Least One Crystallizable Block in the Backbone:

These polymers are notably block copolymers constituted of at least 2 blocks of different chemical nature, one of which is crystallizable.

The block copolymers defined in U.S. Pat. No. 5,156,911 can be used;
Block copolymers of olefin or of cycloolefin with crystallizable chain such as those resulting from the block polymerization of:
   cyclobutene, cyclohexene, cyclooctene, norbornene (i.e. bicyclo(2,2,1)heptene-2), 5-methylnorbornene, 5-ethylnorbornene, 5,6-dimethylnorbornene, 5,5,6-trimethyl norbornene, 5-ethylidene-norbornene, 5-phenyl-norbonene, 5-benzylnorbornene, 5-vinyl norbornene, 1,4,5,8-dimethano-1,2,3,4,4a,5,8a-octahydronaphthalene, dicyclopentadiene or mixtures thereof,
   with ethylene, propylene, 1-butene, 3-methyl-1-butene, 1-hexene, 4-methyl-1-pentene, 1-octene, 1-decene, 1-eicosene or mixtures thereof,
   and in particular the (ethylene/norbornene) block copolymers and the (ethylene/propylene/ethylidene-norbornene) block terpolymers. It is also possible to use those resulting from the block copolymerization of at least 2α-olefins of C₂-C₁₆ and preferably of C₂-C₁₂ and more preferably of C₄-C₁₂ such as those mentioned previously and in particular the diblock copolymers of ethylene and of 1-octene.
The copolymers can be copolymers having at least one crystallizable block, the rest of the copolymer being amorphous (at room temperature). These copolymers can, moreover, have two crystallizable blocks of different chemical nature. The preferred copolymers are those that have, simultaneously at room temperature, a crystallizable block and an amorphous block both hydrophobic and lipophilic distributed sequentially; we may mention for example the polymers possessing one of the following crystallizable blocks and one of the following amorphous blocks:
Naturally crystallizable block: a) polyester such as poly(alkylene terephthalate), b) polyolefin such as polyethylenes or polypropylenes.
Amorphous and lipophilic block such as amorphous polyolefins or copoly(olefin)s such as poly(isobutylene), hydrogenated polybutadiene, hydrogenated poly(isoprene).

As examples of said copolymers with different crystallizable block and amorphous block, we may mention:

α) the poly(ε-caprolactone)-b-poly(butadiene) block copolymers, preferably used hydrogenated, such as those described in the article “Melting behavior of poly(ε-caprolactone)-block-polybutadiene copolymers” by S. Nojima, Macromolécules, 32, 3727-3734 (1999).

β) the hydrogenated poly(butyleneterephthalate)-b-poly(isoprene) block or multiblock copolymers, mentioned in the article “Study of morphological and mechanical properties of PP/PBT” by B. Boutevin et al., Polymer Bulletin, 34, 117-123 (1995).

γ) the poly(ethylene)-b-copoly(ethylene/propylene) block copolymers mentioned in the articles “Morphology of semicrystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997).

δ) the poly(ethylene)-b-poly(ethylethylene) block copolymers mentioned in the general article “Crystallization in block copolymers” by I. W. Hamley, Advances in Polymer Science, Vol. 148, 113-137 (1999).

The semicrystalline polymers of the composition of the invention may or may not be partially crosslinked provided that the degree of crosslinking does not interfere with their dissolution or dispersion in the liquid oily phase by heating above their melting point. It can then be a chemical crosslinking, by reaction with a multifunctional monomer during polymerization. It can also be physical crosslinking, which can then be due either to the establishment of hydrogen or bipolar bonds between groups carried by the polymer, for example bipolar interactions between carboxylate ionomers, said interactions being of small amount and carried by the backbone of the polymer; or with phase separation between the crystallizable blocks and the amorphous blocks, carried by the polymer.

Preferably, the semicrystalline polymers of the composition according to the invention are noncrosslinked.

According to a particular embodiment of the invention, the polymer is selected from the copolymers resulting from the polymerization of at least one monomer with crystallizable chain selected from saturated C₁₄ to C₂₄ alkyl (meth)acrylates, C₁₁ to C₁₅ perfluoroalkyl (meth)acrylates, C₁₄ to C₂₄ N-alkyl (meth)acrylamides with or without a fluorine atom, vinyl esters with C₁₄ to C₂₄ alkyl or perfluoroalkyl chains, vinyl ethers with C₁₄ to C₂₄ alkyl or perfluoroalkyl chains, C₁₄ to C₂₄ alpha-olefins, para-alkyl styrenes with an alkyl group having from 12 to 24 carbon atoms, with at least one ester or amide of monocarboxylic acid of C₁ to C₁₀ optionally fluorinated, which can be represented by the following formula (ω):

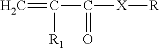

in which R₁ is H or CH₃, R represents an optionally fluorinated C₁-C₁₀ alkyl group and X represents O, NH or NR₂, where R₂ represents an optionally fluorinated C₁-C₁₀ alkyl group.

According to a more particular embodiment of the invention, the polymer is derived from a monomer with crystallizable chain selected from the saturated C₁₄ to C₂₂ alkyl (meth)acrylates and even more particularly poly(stearyl acrylate) or poly(behenyl acrylate).

As particular examples of semicrystalline structure-forming polymer usable in the composition according to the invention, we may mention the polymers having the INCI name “POLY C10-30 ALKYL ACRYLATE such as the Intelimer® products from the company AIR PRODUCTS such as the product Intelimer® IPA 13-1 which is a polystearyl acrylate or the product Intelimer® IPA 13-6 which is a behenyl polymer.

The semicrystalline polymers can notably be:

those described in examples 3, 4, 5, 7, 9, 13 of U.S. Pat. No. 5,156,911 with —COOH group, resulting from the copolymerization of acrylic acid and C₅ to C₁₆ alkyl(meth)acrylate and more particularly from the copolymerization:
of acrylic acid, hexadecylacrylate and isodecylacrylate in the weight ratio 1/16/3,
of acrylic acid and pentadecylacrylate in the weight ratio 1/19,
of acrylic acid, hexadecylacrylate, ethylacrylate in the weight ratio 2.5/76.5/20,
of acrylic acid, hexadecylacrylate and methylacrylate in the weight ratio 5/85/10,
of acrylic acid and octadecylmethacrylate in the weight ratio 2.5/97.5,
of hexadecylacrylate, monomethyl ether of polyethylene glycol methacrylate with 8 ethylene glycol units, and acrylic acid in the weight ratio 8.5/1/0.5.

It is also possible to use the polymer of structure “O” from National Starch such as that described in document U.S. Pat. No. 5,736,125 of melting point 44° C. as well as the semicrystalline polymers with crystallizable pendant chains having fluorinated groups as described in examples 1, 4, 6, 7 and 8 of document WO-A-01/19333.

It is also possible to use the semicrystalline polymers obtained by copolymerization of stearyl acrylate and acrylic acid or NVP as described in document U.S. Pat. No. 5,519,063 or EP-A-550745, with melting point of 40° C. and 38° C. respectively.

It is also possible to use the semicrystalline polymers obtained by copolymerization of behenyl acrylate and acrylic acid or NVP as described in documents U.S. Pat. No. 5,519,063 and EP-A-550745, with melting point of 60° C. and 58° C. respectively.

Preferably, the semicrystalline polymers do not have a carboxyl group.

Finally, the semicrystalline polymers according to the invention can also be selected from the waxy polymers obtained by metallocene catalysis, such as those described in application US2007/0031361.

These polymers are homopolymers or copolymers of ethylene and/or propylene prepared by metallocene catalysis, i.e. by polymerization at low pressure and in the presence of a metallocene catalyst.

The weight-average molecular weight (Mw) of the waxes obtained by metallocene catalysis described in this document is less than or equal to 25 000 g/mol, for example in the range from 2000 to 22 000 g/mol and preferably from 4000 to 20 000 g/mol.

The number-average molecular weight (Mn) of the waxes obtained by metallocene catalysis described in this document is preferably less than or equal to 15 000 g/mol, for example in the range from 1000 to 12 000 g/mol, and preferably from 2000 to 10 000 g/mol.

The polydispersity index I of the polymer is equal to the ratio of the weight-average molecular weight Mw to the number-average molecular weight Mn. Preferably, the polydispersity index of the waxy polymers is between 1.5 and 10, preferably between 1.5 and 5, preferably between 1.5 and 3 and more preferably between 2 and 2.5.

The waxy homopolymers and copolymers can be obtained in a known manner from ethylene and/or propylene monomers for example by metallocene catalysis according to the method described in document EP 571 882.

The homopolymers and copolymers of ethylene and/or propylene prepared by metallocene catalysis can be unmodified or polar modified waxes, i.e. waxes modified so that they have the properties of a polar wax. The polar modified waxy homopolymers and copolymers can be prepared in a known manner from unmodified waxy homopolymers and copolymers such as those described previously by oxidation with gases containing oxygen, such as air, or by grafting with polar monomers such as maleic acid or acrylic acid or derivatives of these acids. These two routes for polar modification of the polyolefins obtained by metallocene catalysis are described respectively in documents EP890 583 and U.S. Pat. No. 5,998,547 for example, the contents of these two documents being incorporated as reference.

According to the present invention, the homopolymers and copolymers of ethylene and/or propylene prepared by metallocene catalysis and polar modified, which are particularly preferred, are the polymers modified so that they have hydrophilic properties. As examples, we may mention homopolymers or copolymers of ethylene and/or propylene modified with the presence of hydrophilic groups such as maleic anhydride, acrylate, methacrylate, polyvinylpyrrolidone (PVP), etc.

The waxy homopolymers or copolymers of ethylene and/or propylene modified with the presence of hydrophilic groups such as maleic anhydride or acrylate are particularly preferred.

As examples, we may mention:
the polypropylene waxes modified with maleic anhydride (PPMA) marketed by the company Clariant or the polypropylene-ethylene-maleic anhydride copolymers, such as those marketed by the company Clariant under the name LicoCare such as LicoCare PP207 LP3349, LicoCare CM401 LP3345, LicoCare CA301 LP 3346, and LicoCare CA302 LP 3347, or
the unmodified polyethylene waxes marketed by the company Clariant, such as the product LicoCare PE 102 LP3329.

Within the scope of a composition for the lips, a polar modified waxy polymer having a low degree of crystallinity, preferably of less than 40%, will be preferred.

(2) Esters of Dextrin and Fatty Acid:

The esters of dextrin and fatty acids can notably be selected from the mono- or poly-esters of dextrin and at least one fatty acid, and the compounds corresponding to formula (III):

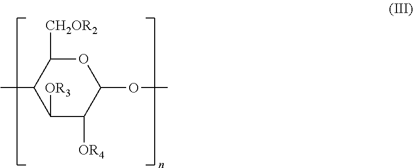

in which:
n is an integer in the range from 3 to 200, notably in the range from 20 to 150, and in particular in the range from 25 to 50,
the radicals R₂, R₃ and R₄, which may be identical or different, are selected from hydrogen or an acyl group (R—CO—) in which the radical R is a hydrocarbon group, linear or branched, saturated or unsaturated, possessing from 7 to 29, in particular from 7 to 21, notably from 11 to 19, more particularly from 13 to 17, or even 15, carbon atoms, provided that at least one of said radicals R₂, R₃ or R₄ is different from hydrogen.

In particular, R₂, R₃ and R₄ can represent hydrogen or an acyl group (R′—CO—) in which R′ is a hydrocarbon radical as defined previously, provided that at least two of said radicals R₂, R₃ or R₄ are identical and different from hydrogen.

Together, the radicals R₂, R₃ and R₄ can represent an acyl group (R′—CO), identical or different, and notably identical.

In particular, n is advantageously from 25 to 50, and notably is equal to 38 in general formula (III) of the ester according to the invention.

Notably when the radicals R₂, R₃ and/or R₄, which may be identical or different, represent an acyl group (R′—CO), the latter can be selected from the caprylic, capric, lauric, myristic, palmitic, stearic, arachic, behenic, isobutyric, isovaleric, ethyl-2-butyric, ethylmethylacetic, isoheptanoic, ethyl-2-hexanoic, isononanoic, isodecanoic, isotridecanoic, isomyristic, isopalmitic, isostearic, isoarachic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic, stearolic radicals, and mixtures thereof.

Preferably, at least one dextrin palmitate is used as ester of dextrin and fatty acid(s). This can be used alone or mixed with other esters.

Advantageously, the ester of dextrin and fatty acid has a degree of substitution less than or equal to 2.5 based on a glucose unit, notably in the range from 1.5 to 2.5, preferably from 2 to 2.5. The weight-average molecular weight of the dextrin ester can be in particular from 10 000 to 150 000, notably from 12 000 to 100 000 and even from 15 000 to 80 000.

Dextrin esters, in particular dextrin palmitates, are available commercially under the designation RHEOPEARL TL or RHEOPEARL KL from the company Chiba Flour.

(3) Modified Hydrophobic Polysaccharides

The polysaccharide used in the present invention is preferably selected from the fructans.

The fructans or fructosans are oligosaccharides or polysaccharides comprising a chain of anhydrofructose units optionally combined with one or more saccharide residues different from fructose. The fructans can be linear or branched. The fructans can be products that are obtained directly from a vegetable or microbial source or products whose chain length has been modified (increased or decreased) by fractionation, synthesis or hydrolysis, in particular enzymatic. The fructans generally have a degree of polymerization from 2 to about 1000 and preferably from 2 to about 60.

The fructans are classified into 3 groups. The first group corresponds to products whose fructose units are for the most part bound by β-2-1 bonds. They are essentially linear fructans such as the inulins. The second group also corresponds to linear fructoses but the fructose units are essentially bound by β-2-6 bonds. These products are levans. The third group corresponds to mixed fructans, i.e. having β-2-6 and β-2-1 chains. They are essentially branched fructans such as the graminans.

The fructans used in the compositions according to the invention are inulins. Inulin can be obtained for example from chicory, dahlia or Jerusalem artichoke. Preferably, the inulin used in the composition according to the invention is obtained for example from chicory.

The polysaccharides, in particular the inulins, used in the compositions according to the invention are modified hydrophobic. In particular, they are obtained by grafting hydrophobic chains onto the hydrophilic backbone of fructan.

The hydrophobic chains that can be grafted on the main chain of the fructan can notably be linear or branched, saturated or unsaturated hydrocarbon chains, having from 1 to 50 carbon atoms, such as the alkyl, aralkyl, alkaryl, alkylene groups; divalent cycloaliphatic groups or organopolysiloxane chains. These hydrocarbon chains or organopolysiloxanes can notably comprise one or more ester, amide, urethane, carbamate, thiocarbamate, urea, thio-urea, and/or sulphonamide functions such as notably methylenedicyclohexyl and isophorone; or divalent aromatic groups such as phenylene.

In particular, the polysaccharide, notably inulin, has a degree of polymerization from 2 to about 1000 and preferably from 2 to about 60, and a degree of substitution less than 2 based on a fructose unit.

According to a preferred embodiment, the hydrophobic chains have at least one alkyl carbamate group of formula R″—NH—CO— in which R″ is an alkyl group having from 1 to 22 carbon atoms.

According to a more preferred embodiment, the hydrophobic chains are lauryl carbamate groups.

In particular, by way of nonlimiting illustration of the modified hydrophobic inulins that can be used in the compositions according to the invention, we may mention stearoyl inulin such as those sold under the names Lifidrem INST by the company Engelhard and Rheopearl INS by the company Ciba; palmitoyl inulin; undecylated inulin such as those sold under the names Lifidrem INUK and Lifidrem INUM by the company Engelhard; and the lauryl carbamate inulin such as that sold under the name INUTEC SP1 by the company ORAFTI.

In particular, the modified hydrophobic polysaccharide is a lauryl carbamate grafted inulin, notably obtained from the reaction of lauryl isocyanate on an inulin, in particular derived from chicory. As examples of these compounds, we may notably mention the product sold under the name INUTEC SP1 by the company ORAFTI.

(4) Crystalline Copolymers of Olefins:

The crystalline olefin copolymers used in the compositions of the present application can be any olefin copolymer, i.e. a copolymer comprising only olefinic units, having a controlled and moderated crystalline character, i.e. a degree of crystallinity at most equal to 50%, preferably between 5 and 40%, and more preferably between 10 and 35%.

These copolymers are generally elastomers or plastomers and can be synthesized by any known method, in particular by a radical route, by Ziegler-Natta catalysis or by metallocene catalysis, preferably by metallocene catalysis.

A first class of crystalline olefin copolymers, usable in the compositions according to the invention, are the copolymers of α-olefin, in particular of α-olefin of C₂-C₁₆ and more preferably of C₂-C₁₂. Preferably, these copolymers are bi- or terpolymers and quite particularly bipolymers.

Among the bipolymers recommended for the compositions of the invention, we may mention the bipolymers of ethylene and of α-olefin of C₄-C₁₆, preferably of C₄-C₁₂ and the bipolymers of propylene and of α-olefin of C₄-C₁₆, preferably of C₄-C₁₂. More preferably, the α-olefin is selected from butene-1, pentene-1, hexene-1, octene-1, nonene-1, decene-1, undecene-1, dodecene-1,3,5,5-trimethylhexene-1,3-methylpentene-1, and 4-methylpentene-1.

Among these monomers, butene-1 and octene-1 are particularly preferred.

The proportion of α-olefin in the bipolymer is generally between 2 and 40 mol. %, preferably 3 to 30 mol. %, and more preferably 4 to 20 mol. %.

The recommended ethylene-octene bipolymers are plastomers having an octene content between 5.2% and 6.2 mol. %, a degree of crystallinity between 28 and 38%, and elastomers having an octene content between 8 and 14 mol. % and a degree of crystallinity between 10 and 28%.

These bipolymers are synthesized by metallocene catalysis.

Said bipolymers are marketed by the company DOW CHEMICAL under the trade names AFFINITY (plastomers) and ENGAGE (elastomers).

Ethylene-butene bipolymers are marketed by the company EXXON under the trade name EXACT RESINS.

Among the terpolymers, we may mention the terpolymers of ethylene, of propylene and of α-olefin of C₄-C₁₆, preferably C₄-C₁₂.

In these terpolymers, the contents of C₄-C₁₆ α-olefin are as stated previously and the preferred α-olefins are butene, hexene and octene.

A second class of copolymers of olefins suitable for the compositions according to the invention are the copolymers of ethylene or of propylene and a cycloolefin, in particular the bipolymers.

Generally, the content of cycloolefin in the copolymers is less than 20 mol. %.

Among the cycloolefins that can be used, we may mention cyclobutene, cyclohexene, cyclooctadiene, norbornene, dimethano-octahydronaphthalene (DMON), ethylidene norbornene, vinyl norbornene and 4-vinylcyclohexene.

The recommended copolymers of this class are the copolymers of ethylene and norbornene. The norbornene content of these copolymers is generally less than 18 mol. % in order to have the required crystalline character and these copolymers are synthesized by metallocene catalysis.

Suitable ethylene/norbornene copolymers are marketed by the companies MITSUI PETROCHEMICAL or MITSUI-SEKKA under the trade name APEL and by the company HOECHST-CELANESE under the trade name TOPAS.

Other recommended ethylene/cycloolefin copolymers are the ethylene/cyclobutene and ethylene/cyclohexene bipolymers with low content of cycloolefin, generally less than 20 mol. %.

A third class of suitable copolymers of olefins is constituted of the copolymers of olefins with controlled tacticity, i.e. copolymers having units with different tacticity.

Among these copolymers with controlled tacticity, we may mention the isotactic propylene/atactic propylene copolymers and syndiotactic propylene/atactic propylene copolymers.

The iso- or syndiotactic units or blocks endow the copolymer with crystalline character, while the amorphous atactic units or blocks prevent excessive crystallinity of the copolymer and control the degree of crystallinity as well as the morphology and size of the crystallites.

The content of iso- or syndiotactic units, which impart the crystalline character to the copolymer, is therefore determined so as to obtain the desired percentage crystallinity 50%) in the copolymer.

The content of tactic units is generally between 10 and 80 mol. %. However, the content of atactic units is preferably less than 30 mol. %.

These copolymers are synthesized by metallocene catalysis.

A fourth class of copolymers of olefins suitable for the present invention is constituted of the copolymers of monoolefin and diene, for example the ethylene/butadiene, propylene/butadiene, ethylene/isoprene and propylene/isoprene bipolymers, and the ethylene/propylene/diene terpolymers, also obtained by metallocene synthesis.

The proportion of diene units in the copolymer with controlled crystallization is generally between 3 and 20 mol. %.

For better control of the crystallinity of the copolymer, we can optionally add, to the composition according to the invention, additives that interfere with crystallization and promote the formation of small crystals. These additives, although used in a small proportion, constitute numerous small nucleation sites, distributed uniformly in the bulk. These additives are typically crystals of an organic or mineral substance.

In the case of an organic additive for crystallization, it must have a melting point above the melting range of the copolymer and should preferably form small crystals.

At a temperature above its melting point, this substance is preferably soluble in the mixture of liquid oily phase and molten polymer. Thus, during cooling, the additive that was initially dissolved, recrystallizes in the form of numerous small crystals, well distributed in the mixture, then the polymer recrystallizes giving small crystal domains owing to the presence of the crystals of additives. This is a conventional technique for recrystallization of polymers.

The degree of crystallization, the size and the morphology of the copolymers of olefins according to the invention can also be adjusted by mixing a first copolymer of olefins according to the invention with a second crystalline polymer or copolymer, partially compatible with the first copolymer of olefins. The second polymer or copolymer can be a copolymer of olefins according to the invention, but with a degree of crystallinity different from that of the first copolymer, including a degree of crystallinity higher than the degree of crystallinity of the copolymers of olefins according to the invention.

The second crystallizable polymer can also be a polymer of a different nature, for example a copolyethylene/vinyl acetate obtained by radical copolymerization or even a crystallizable polyethylene such as those usually employed in the area of cosmetics.

For more detail regarding this method of adjusting the degree of crystallinity, reference may be made to the articles with the titles “Elastomeric blends of homogeneous ethylene-octene copolymers” S. Bensason et al., Polymer, Vol. 38, No. 15, 1997, pages 3913-19, and “Blends of homogeneous ethylene-octene copolymers” S. Bensason et al., Polymer, Vol. 38, No. 14, 1997, pages 3513-20.

(5) Crystalline Polycondensates:

The polycondensate that can be used can be obtained by reaction:
of 10 to 30 wt. %, relative to the total weight of the polycondensate, of at least one polyol comprising 3 to 6 hydroxyl groups;
of 30 to 80 wt. %, relative to the total weight of the polycondensate, of at least one non-aromatic, saturated or unsaturated, linear, branched and/or cyclic monocarboxylic acid, comprising 6 to 32 carbon atoms;
of 0.1 to 10 wt. %, relative to the total weight of the polycondensate, of at least one aromatic monocarboxylic acid comprising 7 to 11 carbon atoms, optionally moreover substituted with 1 to 3, saturated or unsaturated, linear, branched and/or cyclic alkyl radicals, which comprise 1 to 32 carbon atoms;
of 5 to 40 wt. %, relative to the total weight of the polycondensate, of at least one saturated or unsaturated, or even aromatic, linear, branched and/or cyclic polycarboxylic acid, comprising at least 2 carboxyl groups COOH, notably 2 to 4 groups COOH; and/or a cyclic anhydride of said polycarboxylic acid.

Preferably, the polycondensate can be obtained by reaction:
of 10 wt. % of at least one aromatic monocarboxylic acid comprising 7 to 11 carbon atoms, optionally moreover substituted with 1 to 3 saturated or unsaturated, linear, branched and/or cyclic alkyl radicals, which comprise 1 to 32 carbon atoms; and
of 15 to 30 wt. %, relative to the total weight of the polycondensate, of at least one polyol comprising 3 to 6 hydroxyl groups; and
of 30 to 40 wt. %, relative to the total weight of the polycondensate, of at least one non-aromatic, saturated or unsaturated, linear, branched and/or cyclic monocarboxylic acid, comprising 6 to 32 carbon atoms; and
of 10 to 25 wt. %, relative to the total weight of the polycondensate, of at least one saturated or unsaturated, or even aromatic, linear, branched and/or cyclic polycarboxylic acid, comprising at least 2 carboxyl groups COOH, notably 2 to 4 groups COOH; and/or a cyclic anhydride of said polycarboxylic acid; these conditions being cumulative,
   then the ratio of the number of moles of aromatic monocarboxylic acid to the number of moles of non-aromatic monocarboxylic acid is between 0.08 and 0.70.

The polycondensate can also be obtained by reaction:
of 10 to 30 wt. %, relative to the total weight of the polycondensate, of at least one polyol comprising 3 to 6 hydroxyl groups;
of 45 to 80 wt. %, relative to the total weight of the polycondensate, of at least one saturated, linear, branched and/or cyclic non-aromatic monocarboxylic acid, comprising 6 to 32 carbon atoms;
of 0.1 to 10 wt. %, relative to the total weight of the polycondensate, of at least one aromatic monocarboxylic acid comprising 7 to 11 carbon atoms, optionally moreover substituted with 1 to 3 saturated or unsaturated, linear, branched and/or cyclic alkyl radicals, which comprise 1 to 32 carbon atoms;
of 5 to 40 wt. %, relative to the total weight of the polycondensate, of at least one saturated or unsaturated, or even aromatic, linear, branched and/or cyclic polycarboxylic acid, comprising at least 2 carboxyl groups COOH, notably 2 to 4 groups COOH; and/or a cyclic anhydride of said polycarboxylic acid.

One of the constituents required for preparing the polycondensates according to the invention is a compound comprising 3 to 6 hydroxyl groups (polyol), notably 3 to 4 hydroxyl groups. It is of course possible to use a mixture of said polyols. Said polyol can notably be a carbon-containing, notably hydrocarbon compound, linear, branched and/or cyclic, saturated or unsaturated, comprising 3 to 18 carbon atoms, notably 3 to 12, or even 4 to 10 carbon atoms, and 3 to 6 hydroxyl groups (OH), and can moreover comprise one or more oxygen atoms intercalated in the chain (ether function). Said polyol is preferably a saturated, linear or branched hydrocarbon compound, comprising 3 to 18 carbon atoms, notably 3 to 12, or even 4 to 10 carbon atoms, and 3 to 6 hydroxyl groups (OH). It can be selected from, alone or mixed:
triols, such as 1,2,4-butanetriol, 1,2,6-hexanetriol, trimethylolethane, trimethylolpropane, glycerol;
tetraols, such as pentaerythritol (tetramethylolmethane), erythritol, diglycerol or ditrimethylolpropane;
pentols such as xylitol,
hexyls such as sorbitol and mannitol; or dipentaerythritol or triglycerol.

Preferably, the polyol is selected from glycerol, pentaerythritol, diglycerol, sorbitol and mixtures thereof; and more preferably it is pentaerythritol. The polyol, or the polyol mixture, preferably represents 10 to 30 wt. %, notably 12 to 25 wt. %, and more preferably 14 to 22 wt. %, of the total weight of the final polycondensate.

Another constituent required for preparation of the polycondensates according to the invention is a non-aromatic, saturated or unsaturated, linear, branched and/or cyclic monocarboxylic acid, comprising 6 to 32 carbon atoms, notably 8 to 28 carbon atoms and preferably 10 to 24, or even 12 to 20, carbon atoms. It is of course possible to use a mixture of said non-aromatic monocarboxylic acids.

Non-aromatic monocarboxylic acid means a compound of formula R′″COOH, in which R′″ is a saturated or unsaturated, linear, branched and/or cyclic hydrocarbon radical, comprising 5 to 31 carbon atoms, notably 7 to 27 carbon atoms, and preferably 9 to 23 carbon atoms, or even 11 to 19 carbon atoms.

Preferably, the radical R is saturated. More preferably, said radical R is linear or branched, and preferably of C₅-C₃₁, or even of C₁₁-C₂₁.

In a particular embodiment of the invention, the non-aromatic monocarboxylic acid has a melting point greater than or equal to 25° C., notably greater than or equal to 28° C., or even to 30° C.; it was in fact found that when such an acid is used, especially in large amount, it is possible on the one hand to obtain good gloss and durability of said gloss, and on the other hand to reduce the amount of waxes usually present in the composition envisaged.

Among the non-aromatic monocarboxylic acids that can be used, we may mention, alone or mixed:
saturated monocarboxylic acids such as caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic); cyclopentanecarboxylic acid, cyclopentaneacetic acid, 3-cyclopentyl-propionic acid, cyclohexanecarboxylic acid, cyclohexylacetic acid, 4-cyclohexylbutyric acid;
unsaturated but non-aromatic monocarboxylic acids, such as caproleic acid, obtusilic acid, undecylene, dodecylene, linderic acid, myristoleic acid, physeteric acid, tsuzuic acid, palmitoleic acid, oleic acid, petroselinic acid, vaccenic acid, elaidic acid, gondoic acid, gadoleic acid, erucic acid, cetoleic acid, nervonic acid, linoleic acid, linolenic acid, arachidonic acid.

Among the non-aromatic monocarboxylic acids having a melting point greater than or equal to 25° C., we may mention, alone or mixed:
among the saturated monocarboxylic acids: decanoic (capric) acid, lauric acid, tridecanoic acid, myristic acid, palmitic acid, stearic acid, arachidic acid, behenic acid, cerotic (hexacosanoic) acid;
among unsaturated but non-aromatic monocarboxylic acids: petroselinic acid, vaccenic acid, elaidic acid, gondoic acid, gadoleic acid, erucic acid, nervonic acid.

Preferably, it is possible to use 2-ethylhexanoic acid, isooctanoic acid, lauric acid, myristic acid, isoheptanoic acid, isononanoic acid, nonanoic acid, palmitic acid, isostearic acid, stearic acid, behenic acid and mixtures thereof, and more preferably isostearic acid alone or stearic acid alone.

Said non-aromatic monocarboxylic acid, or the mixture of said acids, preferably represents 30 to 80 wt. %, notably 40 to 75 wt. %, or even 45 to 70 wt. %, and more preferably 50 to 65 wt. %, of the total weight of the final polycondensate.

Another constituent required for preparation of the polycondensates according to the invention is an aromatic monocarboxylic acid comprising 7 to 11 carbon atoms, optionally moreover substituted with 1 to 3 saturated or unsaturated, linear, branched and/or cyclic alkyl radicals, which comprise 1 to 32 carbon atoms, notably 2 to 12, or even 3 to 8 carbon atoms. It is of course possible to use a mixture of said aromatic monocarboxylic acids.

Aromatic monocarboxylic acid means a compound of formula R″″COOH, in which R″″ is an aromatic hydrocarbon radical, comprising 6 to 10 carbon atoms, and in particular the radicals benzoic and naphthoic. Said radical R″″ can moreover be substituted with 1 to 3 saturated or unsaturated, linear, branched and/or cyclic alkyl radicals, comprising 1 to 32 carbon atoms, notably 2 to 12, or even 3 to 8 carbon atoms; and notably selected from methyl, ethyl, propyl, isopropyl, butyl, isobutyl, terbutyl, pentyl, isopentyl, neopentyl, cyclopentyl, hexyl, cyclohexyl, heptyl, isoheptyl, octyl or isooctyl. Among the aromatic monocarboxylic acids that can be used, we may mention, alone or mixed, benzoic acid, o-toluic acid, m-toluic acid, p-toluic acid, 1-naphthoic acid, 2-naphthoic acid, 4-tert-butyl-benzoic acid, 1-methyl-2-naphthoic acid, 2-isopropyl-1-naphthoic acid. Preferably, it is possible to use benzoic acid, 4-tert-butyl-benzoic acid, o-toluic acid, m-toluic acid, 1-naphthoic acid, alone or mixed; and more preferably benzoic acid alone. Said aromatic monocarboxylic acid, or mixture of said acids, preferably represents 0.1 to 10 wt. %, notably 0.5 to 9.95 wt. %, more preferably from 1 to 9.5 wt. %, or even 1.5 to 8 wt. %, of the total weight of the final polycondensate.

Another constituent required for preparation of the polycondensates according to the invention is a saturated or unsaturated, or even aromatic, linear, branched and/or cyclic polycarboxylic acid, comprising at least 2 carboxyl groups COOH, notably 2 to 4 groups COOH; and/or a cyclic anhydride of said polycarboxylic acid. It is of course possible to use a mixture of said polycarboxylic acids and/or anhydrides. Said polycarboxylic acid can notably be selected from the linear, branched and/or cyclic, saturated or unsaturated, or even aromatic polycarboxylic acids, comprising 2 to 50, notably 2 to 40, carbon atoms, in particular 3 to 36, or even 3 to 18, and more preferably 4 to 12 carbon atoms, or even 4 to 10 carbon atoms; said acid comprises at least two carboxyl groups COOH, preferably from 2 to 4 groups COOH.

Preferably, said polycarboxylic acid is aliphatic, saturated, and linear and comprises 2 to 36 carbon atoms, notably 3 to 18 carbon atoms, or even 4 to 12 carbon atoms; or else is aromatic and comprises 8 to 12 carbon atoms. It preferably comprises 2 to 4 groups COOH. Said cyclic anhydride of said polycarboxylic acid can notably correspond to one of the following formulae:

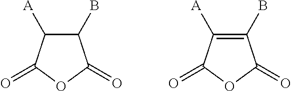

in which the groups A and B are, independently of one another:
a hydrogen atom,
a carbon-containing, aliphatic, saturated or unsaturated, linear, branched and/or cyclic, or else aromatic, radical; comprising 1 to 16 carbon atoms, notably 2 to 10 carbon atoms, or even 4 to 8 carbon atoms, notably methyl or ethyl;
or else A and B taken together form a ring comprising a total of 5 to 7, notably 6 carbon atoms, saturated or unsaturated, or even aromatic.

Preferably, A and B represent a hydrogen atom or together form an aromatic ring comprising a total of 6 carbon atoms.

Among the polycarboxylic acids or their anhydrides that can be used, we may mention, alone or mixed:
dicarboxylic acids such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid, dimers of fatty acids (notably of C₃₆) such as the products marketed under the names Pripol 1006, 1009, 1013 and 1017, by Uniqema;
tricarboxylic acids such as cyclohexanetricarboxylic acid, trimellitic acid, 1,2,3-benzenetricarboxylic acid, 1,3,5-benzenetricarboxylic acid;
tetracarboxylic acids such as butanetetracarboxylic acid and pyromellitic acid,
cyclic anhydrides of these acids and notably phthalic anhydride, trimellitic anhydride, maleic anhydride and succinic anhydride.

Preferably, it is possible to use adipic acid, phthalic anhydride and/or isophthalic acid, and more preferably isophthalic acid alone.

Said polycarboxylic acid and/or its cyclic anhydride preferably represents 5 to 40 wt. %, notably 10 to 30 wt. %, and more preferably 14 to 25 wt. %, of the total weight of the final polycondensate.

The polycondensate according to the invention can moreover comprise a silicone with hydroxyl (OH) and/or carboxyl (COOH) function.

It can comprise 1 to 3 hydroxyl and/or carboxyl functions, and preferably comprises two hydroxyl functions or else two carboxyl functions.

These functions can be positioned at the end of the chain or in the chain, but advantageously at the end of the chain.

Preferably silicones are used having a weight-average molecular weight (Mw) between 300 and 20 000, notably 400 and 10 000, or even 800 and 4000.

Said silicone can be of formula (IV):

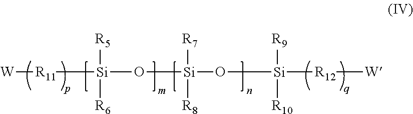

in which:
W and W′ are, independently of one another, OH or COOH; preferably W═W′;
p and q are, independently of one another, equal to 0 or 1,
R₁₁ and R₁₂ are, independently of one another, a carbon-containing, notably hydrocarbon, saturated or unsaturated, or even aromatic, linear, branched and/or cyclic divalent radical; comprising 1 to 12 carbon atoms, notably 2 to 8 carbon atoms, and optionally further comprising one or more heteroatoms selected from O, S and N, notably O (ether);
   notably R¹¹ and/or R₁₂ can be of formula —(CH₂)ₐ— with a=1-12, and notably methylene, ethylene, propylene, phenylene;
   or else of formula —[(CH₂)ₓO]_{z}— with x=1, 2 or 3 and z=1-10; in particular x=2 or 3 and z=1-4; and more preferably x=3 and z=1.
R₅ to R₁₀ are, independently of one another, a carbon-containing linear, branched and/or cyclic, saturated or unsaturated or even aromatic radical; comprising 1 to 20 carbon atoms, notably 2 to 12 carbon atoms; preferably, R₅ to R₁₀ are saturated or else aromatic, and can notably be selected from the alkyl radicals, in particular the methyl, ethyl, propyl, isopropyl, butyl, pentyl, hexyl, octyl, decyl, dodecyl and octadecyl radicals, the cycloalkyl radicals, in particular the cyclohexyl radical, the aryl radicals, notably phenyl and naphthyl, the aralkyl radicals, notably benzyl and phenylethyl, as well as the tolyl and xylyl radicals.
m and n are, independently of one another, integers between 1 and 140, and are such that the weight-average molecular weight (Mw) of the silicone is between 300 and 20 000, notably between 400 and 10 000, or even between 800 and 4000.

We may notably mention the α,ω-diol or α,ω-dicarboxylic polyalkylsiloxanes, and notably the α,ω-diol polydimethysiloxanes and the α,ω-dicarboxylic polydimethylsiloxanes; the α,ω-diol or α,ω-dicarboxylic polyarylsiloxanes and notably the α,ω-diol or α,ω-dicarboxylic polyphenylsiloxanes; the polyarylsiloxanes with silanol functions such as polyphenylsiloxane; the polyalkylsiloxanes with silanol functions such as polydimethylsiloxane; the polyaryl/alkylsiloxanes with silanol functions such as polyphenyl/methylsiloxane or polyphenyl/propylsiloxane.

Quite particularly, the α,ω-diol polydimethysiloxanes with weight-average molecular weight (Mw) between 400 and 10 000, or even between 500 and 5000, and notably between 800 and 4000, will be used.

When it is present, said silicone can preferably represent 0.1 to 15 wt. %, notably 1 to 10 wt. %, or even 2 to 8 wt. %, of the weight of the polycondensate.

In a preferred embodiment of the invention, the aromatic monocarboxylic acid is present in a molar amount less than or equal to that of the non-aromatic monocarboxylic acid; notably the ratio of the number of moles of aromatic monocarboxylic acid to the number of moles of non-aromatic monocarboxylic acid is preferably between 0.08 and 0.70, notably between 0.10 and 0.60, in particular between 0.12 and 0.40.

Preferably, the polycondensate according to the invention can be obtained by reaction:
of at least one polyol selected from, alone or mixed, 1,2,6-hexanetriol, trimethylolethane, trimethylolpropane, glycerol; pentaerythritol, erythritol, diglycerol, ditrimethylolpropane; xylitol, sorbitol, mannitol, dipentaerythritol and/or triglycerol; preferably present in an amount from 10 to 30 wt. %, notably 12 to 25 wt. %, and more preferably 14 to 22 wt. %, relative to the total weight of the final polycondensate;
of at least one non-aromatic monocarboxylic acid selected from, alone or mixed, caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid; cyclopentanecarboxylic acid, cyclopentaneacetic acid, 3-cyclopentylpropionic acid, cyclohexanecarboxylic acid, cyclohexylacetic acid, 4-cyclohexylbutyric acid; preferably present in an amount from 30 to 80 wt. %, notably 40 to 75 wt. %, and more preferably 45 to 70 wt. %, relative to the total weight of the final polycondensate;
of at least one aromatic monocarboxylic acid selected from, alone or mixed, benzoic acid, o-toluic acid, m-toluic acid, p-toluic acid, 1-naphthoic acid, 2-naphthoic acid, 4-tert-butyl benzoic acid, 1-methyl-2-naphthoic acid, 2-isopropyl-1-naphthoic acid; preferably present in an amount from 0.1 to 10 wt. %, notably 1 to 9.5 wt. %, or even 1.5 to 8 wt. %, relative to the total weight of the final polycondensate; and
of at least one polycarboxylic acid or one of its anhydrides, selected from, alone or mixed, decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid; cyclohexanetricarboxylic acid, trimellitic acid, 1,2,3-benzenetricarboxylic acid, 1,3,5-benzenetricarboxylic acid; butanetetracarboxylic acid, pyromellitic acid, phthalic anhydride, trimellitic anhydride, maleic anhydride and succinic anhydride; preferably present in an amount from 5 to 40 wt. %, notably 10 to 30 wt. %, and more preferably 14 to 25 wt. %, relative to the total weight of the final polycondensate.

Preferably, the polycondensate according to the invention can be obtained by reaction:
of at least one polyol selected from, alone or mixed, glycerol, pentaerythritol, sorbitol and mixtures thereof, and more preferably pentaerythritol alone; present in an amount from 10 to 30 wt. %, notably 12 to 25 wt. %, and more preferably 14 to 22 wt. %, relative to the total weight of the final polycondensate;
of at least one non-aromatic monocarboxylic acid selected from, alone or mixed, 2-ethylhexanoic acid, isooctanoic acid, lauric acid, palmitic acid, isostearic acid, isononanoic acid, stearic acid, behenic acid and mixtures thereof, and more preferably isostearic acid alone or stearic acid alone; present in an amount from 30 to 80 wt. %, notably 40 to 75 wt. %, and more preferably 45 to 70 wt. %, relative to the total weight of the final polycondensate;
of at least one aromatic monocarboxylic acid selected from, alone or mixed, benzoic acid, o-toluic acid, m-toluic acid, 1-naphthoic acid, and more preferably benzoic acid alone; present in an amount from 0.1 to 10 wt. %, notably 1 to 9.5 wt. %, or even 1.5 to 8 wt. % relative to the total weight of the final polycondensate; and
of at least one polycarboxylic acid or one of its anhydrides, selected from, alone or mixed, phthalic anhydride and isophthalic acid, and more preferably isophthalic acid alone; present in an amount from 5 to 40 wt. %, notably 10 to 30 wt. %, and more preferably 14 to 25 wt. %, relative to the total weight of the final polycondensate.

The polycondensate according to the invention can be prepared by the methods of esterification/polycondensation usually employed by a person skilled in the art.

By way of illustration, a general method of preparation comprises:
mixing the polyol and the aromatic and non-aromatic monocarboxylic acids,
heating the mixture under inert atmosphere, at first up to the melting point (generally 100-130° C.) and then to a temperature between 150 and 220° C. until the monocarboxylic acids are completely consumed (attained when the acid number is less than or equal to 1), preferably progressively distilling the water that formed, then
optionally cooling the mixture to a temperature between 90 and 150° C.,
adding the polycarboxylic acid and/or the cyclic anhydride, and optionally the silicone with hydroxyl or carboxyl functions, in one go or successively, then
heating again to a temperature less than or equal to 220° C., notably between 170 and 220° C., preferably continuing to remove the water that forms, until the required characteristics in terms of acid number, viscosity, hydroxyl number and solubility are obtained.

It is possible to add conventional esterification catalysts, for example of the sulphonic acid type (notably at a concentration by weight between 1 and 10%) or titanate type (notably at a concentration by weight between 5 and 100 ppm).

It is also possible to carry out the reaction, wholly or partly, in an inert solvent such as xylene and/or at reduced pressure, to facilitate removal of water.

Advantageously, neither catalyst nor solvent is used.

Said method of preparation can further comprise a stage of addition of at least one antioxidant to the reaction mixture, notably at a concentration by weight between 0.01 and 1%, relative to the total weight of monomers, so as to limit any degradation associated with prolonged heating.

The antioxidant can be of the primary type or of the secondary type, and can be selected from hindered phenols, secondary aromatic amines, organophosphorus compounds, sulphur-containing compounds, lactones, acrylated bisphenols; and mixtures thereof.

(6) Lipophilic Mineral Structure-Forming Agents

The rheological agent for thickening or gelling the fatty phase can be a lipophilic mineral structure-forming agent.

We may notably mention the lipophilic clays, such as clays optionally modified such as hectorites modified with an ammonium chloride of C₁₀ to C₂₂ fatty acid, such as hectorite modified with distearyl dimethyl ammonium chloride.

We may also mention the hydrophobic silicas, such as pyrogenic silica optionally with hydrophobic surface treatment whose particle size is less than 1 μm. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:
trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot.
dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot.

Hydrophobic pyrogenic silica preferably has a particle size that can be nanometric to micrometric, for example in the range from about of 5 to 200 nm.

(7) Lipophilic Polyamide Polycondensates

“Polycondensate” means, in the sense of the invention, a polymer obtained by polycondensation, that is by chemical reaction between monomers possessing different functional groups selected in particular from the acid, alcohol and amine functions.

“Polymer” means, in the sense of the invention, a compound having at least 2 repeating units, preferably at least 3 repeating units and more preferably 10 repeating units.

The lipophilic polyamide polycondensate(s) are preferably present in the compositions of the invention at concentrations in the range from 0.1 to 15 wt. % relative to the total weight of the composition, more preferably from 1 to 8 wt. %.

The lipophilic polyamide polycondensates can notably be selected from the polyamide polymers comprising a) a polymer backbone having hydrocarbon repeating units with at least one nonpendant amide unit, and optionally b) at least one pendant fatty chain and/or at least one terminal fatty chain optionally functionalized, comprising at least 4 carbon atoms and bound to these hydrocarbon units.

“Functionalized chains” means, in the sense of the invention, an alkyl chain having one or more functional or reactive groups notably selected from the amide, hydroxyl, ether, oxyalkylene or polyoxyalkylene groups, halogen, including the fluorinated or perfluorinated, ester, siloxane, polysiloxane groups. Moreover, the hydrogen atoms of one or more fatty chains can be substituted at least partially with fluorine atoms.

“Hydrocarbon repeating units” means, in the sense of the invention, a unit having from 2 to 80 carbon atoms, and preferably from 2 to 60 carbon atoms, bearing hydrogen atoms and optionally oxygen atoms, which can be linear, branched or cyclic, saturated or unsaturated. These units each further comprise at least one amide group, advantageously nonpendant and located in the polymer backbone.

Advantageously, the pendant chains are bound directly to at least one of the nitrogen atoms of the polymer backbone.

The lipophilic polyamide polycondensate can contain silicone units or alkoxylated units between the hydrocarbon units.

Moreover, the lipophilic polyamide polycondensate of the composition of the invention advantageously comprises from 40 to 98% of fatty chains relative to the total number of amide units and fatty chains and more preferably from 50 to 95%.

Preferably, the pendant fatty chains are bound to at least one of the nitrogen atoms of the amide units of the polymer. In particular, the fatty chains of this polyamide represent from 40 to 98% of the total number of amide units and fatty chains, and more preferably from 50 to 95%.

Advantageously, the lipophilic polyamide polycondensate has a weight-average molecular weight below 100 000 (notably in the range from 1000 to 100 000), in particular below 50 000 (notably in the range from 1000 to 50 000), and more particularly in the range from 1000 to 30 000, preferably from 2000 to 20 000, and more preferably from 2000 to 10 000.

The lipophilic polyamide polycondensate is water-insoluble, notably at 25° C. In particular, it does not have ionic groups.

As preferred lipophilic polyamide polycondensates for use in the invention, we may mention the polyamides branched with pendant fatty chains and/or terminal fatty chains having from 6 to 120 carbon atoms and more preferably from 8 to 120 and notably from 12 to 68 carbon atoms, each terminal fatty chain being bound to the polyamide backbone by at least one linker L. The linker L can be selected from the ester, ether, amine, urea, urethane, thioester, thioether, thiurea, thiourethane groups. Preferably, these polymers have a fatty chain at each end of the polyamide backbone.

These polymers are preferably polymers resulting from a polycondensation between a carboxylic diacid having at least 32 carbon atoms (notably having from 32 to 44 carbon atoms) with an amine selected from the diamines having at least 2 carbon atoms (notably from 2 to 36 carbon atoms) and the triamines having at least 2 carbon atoms (notably from 2 to 36 carbon atoms). The diacid is preferably a dimer derived from a fatty acid with ethylenic unsaturation having at least 16 carbon atoms, preferably from 16 to 24 carbon atoms, such as oleic, linoleic or linolenic acid. The diamine is preferably ethylene diamine, hexylene diamine, hexamethylene diamine. The triamine is for example ethylene triamine. For the polymers having 1 or 2 terminal carboxylic acid groups, it is advantageous to esterify them with a monohydric alcohol having at least 4 carbon atoms, preferably from 10 to 36 carbon atoms and more preferably from 12 to 24 and most preferably from 16 to 24, for example 18 carbon atoms.

The lipophilic polyamide polycondensate of the composition according to the invention can in particular be selected from the polymers of the following formula (V):

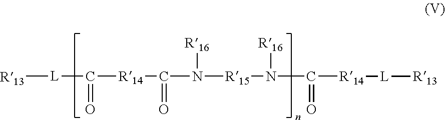

in which:
n is an integer in the range from 1 to 30,
R′₁₃ represents, independently wherever it occurs, a fatty chain and is selected from an alkyl or alkenyl group having at least 1 carbon atom and notably from 4 to 24 carbon atoms;
R′₁₄ represents, independently wherever it occurs, a hydrocarbon radical comprising 1 to 52 carbon atoms;
R′₁₅ represents, independently wherever it occurs, an organic group comprising at least one atom selected from the atoms of carbon, hydrogen, or nitrogen, with the proviso that R′₁₅ comprises at least 3 carbon atoms;
R′₁₆ represents, independently wherever it occurs: a hydrogen atom, an alkyl group comprising 1 to 10 carbon atoms, or a direct bond to at least one group selected from R′₁₅ and another R′₁₆ so that when said group is another R′₁₆, the nitrogen atom to which both R′₁₅ and R′₁₆ are bound forms part of a heterocyclic structure defined by R′₁₆—N—R′₁₅, with the proviso that at least 50% of the R′₄ represent a hydrogen atom, and L represents a linker preferably selected from ester, ether, amine, urea, urethane, thioester, thioether, thiurea, thiourethane, optionally substituted with at least one group R′₁ as defined above.

According to one embodiment, these polymers are selected from the polymers of formula (V) in which the linker L represents an ester group

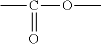

These polymers are more especially those described in document U.S. Pat. No. 5,783,657 of the company Union Camp.

Each of these polymers notably satisfies the following formula (B):

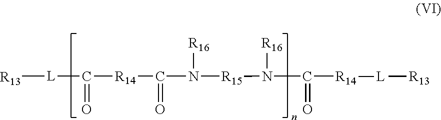

in which:
m denotes a whole number of amide units such that the number of ester groups represents from 10% to 50% of the total number of the ester and amide groups;
R₁₃ is, independently wherever it occurs, an alkyl or alkenyl group having at least 4 carbon atoms and notably from 4 to 24 carbon atoms;
R₁₄ represents, independently wherever it occurs, a C₄ to C₄₂ hydrocarbon group provided that 50% of the groups R₁₄ represent a C₃₀ to C₄₂ hydrocarbon group;
R₁₅ represents, independently wherever it occurs, an organic group with at least 2 carbon atoms, hydrogen atoms and optionally one or more oxygen or nitrogen atoms;
and R₁₆ represents, independently wherever it occurs, a hydrogen atom, a C₁ to C₁₀ alkyl group or a direct bond to R₁₅ or to another R₁₆ so that the nitrogen atom to which R₃ and R₄ are both bound forms part of a heterocyclic structure defined by R₁₆—N—R₁₅, with at least 50% of the R₄ representing a hydrogen atom.

In the particular case of formula (VI), the terminal fatty chains optionally functionalized in the sense of the invention are terminal chains bound to the last nitrogen atom of the polyamide backbone.

In particular, the ester groups of formula (VI), which form part of the terminal and/or pendant fatty chains in the sense of the invention, represent from 15 to 40% of the total number of the ester and amide groups and more preferably from 20 to 35%.

Moreover, m advantageously represents an integer in the range from 1 to 5 and more preferably greater than 2.

Preferably, R₁₃ is a C₁₂ to C₂₂ and preferably C₁₆ to C₂₂ alkyl group. Advantageously, R₁₄ can be a C₁₀ to C₄₂ hydrocarbon (alkylene) group. Preferably, at least 50% and more preferably at least 75% of the R₂ are groups having from 30 to 42 carbon atoms. The other R₂ are hydrogenated groups of C₄ to C₁₉ and even of C₄ to C₁₂.

Preferably, R₁₅ represents a C₂ to C₃₆ hydrocarbon group or a polyalkoxylated group and R₁₆ represents a hydrogen atom. Preferably, R₁₅ represents a C₂ to C₁₂ hydrocarbon group.

The hydrocarbon groups can be linear, cyclic or branched, saturated or unsaturated groups. Moreover, the alkyl and alkylene groups can be linear or branched, saturated or unsaturated groups.

In general, the polymers of formula (VI) are in the form of polymer blends, and moreover these blends can contain a product of synthesis corresponding to a compound of formula (VI) where n has the value 0, i.e. a diester.

According to a particularly preferred embodiment of the invention, a mixture of copolymers of a C₃₆ diacid condensed on ethylene diamine will be used; the terminal ester groups result from the esterification of the residual acid terminations by the cetyl or stearyl alcohol or mixtures thereof (also called cetylstearyl alcohol) (INCI name: ETHYLENEDIAMINE/STEARYL DIMER DILINOLEATE COPOLYMER). Its weight-average molecular weight is preferably 6000. These mixtures are notably sold by the company ARIZONA CHEMICAL under the trade names UNICLEAR 80 and UNICLEAR 100 VG respectively in the form of gel at 80% (of active substance) in a mineral oil and at 100% (of active substance). They have a softening point from 88° C. to 94° C.

As polyamide polycondensates corresponding to general formula (VI), we may also mention the polymers comprising at least one terminal fatty chain bound to the polymer backbone by at least one tertiary amide binding group (also called amide terminated polyamide or ATPA). For more information about these polymers, reference may be made to document U.S. Pat. No. 6,503,522.

According to a particularly preferred embodiment of the invention, more particularly a copolymer of hydrogenated linoleic diacid, of ethylenediamine, of di(C₁₄-C₁₈)alkylamine(s) (INCI name: ETHYLENEDIAMIDE/HYDROGENATED DIMER DILINOLEATE COPOLYMER BIS-DI-C₁₄-C₁₈ ALKYL AMIDE) will be used. This copolymer is notably sold under the trade name SYLVACLEAR A200V by the company ARIZONA CHEMICAL.

According to another embodiment, the polyamide of formula (A) can also be a poly(ester-amide) with ester ends (ester-terminated poly(ester-amide) or ETPEA), for example those whose preparation is described in document U.S. Pat. No. 6,552,160.

According to a particularly preferred embodiment of the invention, more particularly a copolymer of hydrogenated linoleic diacid, of ethylenediamine, of neopentylglycol and of stearyl alcohol (INCI name: BIS-STEARYL ETHYLENEDIAMINE/NEOPENTYL GLYCOL/STEARYL HYDROGENATED DIMER DILINOLEATE COPOLYMER) will be used. This copolymer is notably sold under the trade name SYLVACLEAR C75 V by the company ARIZONA CHEMICAL.

As polyamide polycondensates usable in the invention, we may also mention those comprising at least one terminal fatty chain bound to the polymer backbone by at least one ether or polyether binding group (it is then called ether terminated poly(ether)amide). Said polymers are described for example in document U.S. Pat. No. 6,399,713.

The polyamides according to the invention advantageously have a softening point above 65° C. and which can be up to 190° C. Preferably, it has a softening point in the range from 70 to 130° C. and more preferably from 80 to 105° C. The polyamide is in particular a non-waxy polymer.

As polyamide polycondensates usable in the invention, we may also mention the polyamide resins resulting from the condensation of an aliphatic dicarboxylic acid and a diamine (including the compounds having more than 2 carbonyl groups and 2 amine groups), the carbonyl and amine groups of adjacent individual units being condensed by an amide bond. These polyamide resins are notably those marketed under the brand name Versamid® by the companies General Mills, Inc. and Henkel Corp. (Versamid 930, 744 or 1655) or by the company Olin Mathieson Chemical Corp., under the brand name Onamid® notably Onamid S or C. These resins have a weight-average molecular weight in the range from 6000 to 9000. For more information about these polyamides, reference may be made to documents U.S. Pat. No. 3,645,705 and U.S. Pat. No. 3,148,125. More especially, Versamid® 930 or 744 is used.

It is also possible to use the polyamides sold by the company Arizona Chemical under the references Uni-Rez (2658, 2931, 2970, 2621, 2613, 2624, 2665, 1554, 2623, 2662) and the product sold under the reference Macromelt 6212 by the company Henkel. For more information about these polyamides, reference may be made to document U.S. Pat. No. 5,500,209.

It is also possible to use polyamide resins derived from legumes such as those described in U.S. Pat. No. 5,783,657 and U.S. Pat. No. 5,998,570.

(8) Lipophilic Polyurea or Polyurethane Polymers

As rheological agent for the fatty phase, we may also mention the polyurethanes and polyureas that are soluble or dispersible in hydrocarbon oil(s), and comprising:
at least two urethane groups, or at least two urea groups or at least one urethane group and one urea group in the chain,
at least one hydrocarbon block or graft or aliphatic polyester with a long hydrocarbon chain, preferably branched.

Long hydrocarbon chain means a hydrocarbon chain, linear or branched, having at least 8 carbon atoms and preferably 10 to 500 carbon atoms.

The preferred polymers according to the invention are defined by one of the following three formulae (VII), (VIII) and (IX):

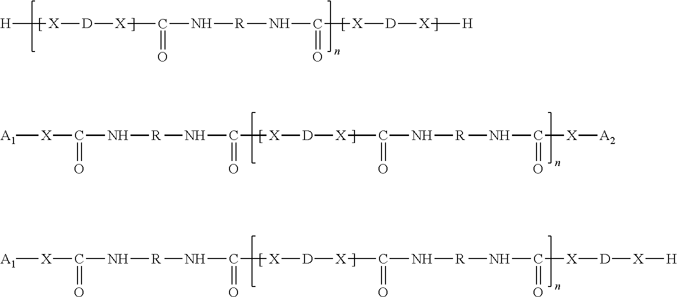

in which:
n denotes an integer from 1 to 10 000, and preferably from 1 to 1000,
x represents, separately or jointly, —O— or —NH—,
R is a divalent radical selected from the alkylene, cycloalkylene, and aromatic radicals, and mixtures thereof, optionally functionalized,
A₁ and A₂, which may be identical or different, denote linear, branched or cyclic monovalent hydrocarbon radicals, saturated or which can have unsaturations, having from 1 to 80 carbon atoms,

### D is

1) a aliphatic, and/or cycloaliphatic saturated or unsaturated divalent hydrocarbon block, and/or aliphatic polyester with a long hydrocarbon chain, or
2) a graft

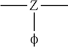

where Z is a trivalent hydrocarbon radical that can contain one or more heteroatoms, and φ is a linear, branched or cyclic aliphatic chain,
3) mixtures of blocks 1) and grafts 2).

The monovalent hydrocarbon radicals A₁ and A₂ are preferably selected from the saturated or unsaturated aliphatic, cycloaliphatic and aromatic radicals. The radicals A₁ and A₂ are derived from the monohydric alcohols and/or monoamines optionally used for consuming the residual isocyanate groups at the end of polymerization.

In the case when D is a saturated or unsaturated aliphatic and/or cycloaliphatic hydrocarbon block, it is derived from:
a natural or synthetic oil, or
the addition product (dimer, trimer or polymer) of at least two unsaturated aliphatic chains, such as the aliphatic radicals derived from “dimeric” fatty acids, for example the products of addition between oleic chains, or
polyenes, preferably hydrogenated, such as polybutadiene, hydrogenated polyisoprene, or polyolefins or copolyolefins.

In the case when D is an aliphatic polyester block with a long hydrocarbon chain, it is preferably derived from branched polyesters with long hydrocarbon chains, for example poly(12-hydroxystearate).

In the case when D is a graft, φ is a linear, branched or cyclic, saturated or unsaturated, aliphatic chain, having from 8 to 40 carbon atoms. The optional heteroatoms of the trivalent radical Z are preferably —O—, —N—, and —S—.

The structure-forming polyurethanes and/or polyureas according to the invention result from the reaction of polymerization between:

1) at least one aliphatic, cycloaliphatic and/or aromatic diisocyanate of general formula O═C═N—R—N═C═O, where R is as defined previously,
2) at least one bifunctional derivative HX-D-XH, having two active hydrogens each of which can react with an isocyanate group, where
X denotes —O— or —NH—, and
D is as defined previously, and
   3) optionally, a monofunctional derivative A₁-XH, or two monofunctional derivatives A₁-XH and A₂-XH, having a single active hydrogen which can react with an isocyanate group, to consume the residual isocyanate groups that would not have reacted fully with the bifunctional reagents H—X-D-X—H, moreover the monofunctional derivatives A₁-XH and A₂-XH can be identical or different, and A₁ and A₂ are as defined previously.

The isocyanates used in the reaction of polymerization can be aliphatic, cycloaliphatic or aromatic. Advantageously, hexamethylene diisocyanate, isophorone diisocyanate, toluene diisocyanate, 4,4′-dicyclohexylmethane diisocyanate will be used.

The bifunctional derivatives H—X-D-X—H can be selected from the dimer diols and their derivatives, the alkane diols, the polydienes with hydroxyl end groups, preferably hydrogenated, the polyolefins with hydroxyl end groups, the branched polyesters with a long alkyl chain bearing at least two reactive groups, the natural or synthetic oils bearing from two to three hydroxyl groups, and finally the dimer diamines and the diamines with a long aliphatic chain.

The dimer diols are branched C₃₆ diols, aliphatic and/or alicyclic, and/or a mixture of said dimers. These diols are prepared from the “corresponding dimeric fatty acids”.

“Corresponding dimeric fatty acids” means the dimeric fatty acids that have the same structure as these diols, but which have two carboxylic acid end groups in place of the diol end groups. The dimeric fatty acids can be converted to dimer diols either by hydrogenation of methyl esters of the dimeric fatty acids, or by direct dimerization of oleic alcohol. We may mention, in particular, the dimer diols sold by the company COGNIS under the trade names SOVERMOL 908 (at 97% purity), and SOVERMOL 650 NS (at 68% purity).

It is also possible to use the polyether-diol oligomers and polycarbonate diols, prepared by subsequent etherification or esterification of these same branched C₃₆ dimer diols. These oligomers generally have a number-average molecular weight of the order of 500 to 2000, and possess two hydroxyl functions.

The polydienes with hydroxyl end groups are for example those defined in French patent FR-2782723. They are selected from the group comprising the homo- and copolymers of polybutadiene, of polyisoprene and of poly(1,3-pentadiene). These oligomers have a number-average molecular weight below 7000, and preferably from 1000 to 5000. They have an end-of-chain functionality from 1.8 to 3, and preferably of about 2. These polydienes with hydroxyl end groups are for example the hydroxylated polybutadienes marketed by the company ELF ATOCHEM under the brand names POLY BD-45H® and POLY BD R-20 LM®. These products are preferably used in hydrogenated form.

It is also possible to use polyolefins, homopolymers or copolymers, with α,ω hydroxyl end groups, for example:
the oligomers of polyisobutylene with α,ω hydroxyl end groups, or
the copolymers marketed by the company MITSUBISHI under the brand name POLYTAIL®, notably those of the following formula (X):

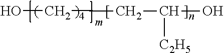

having a melting point from 60 to 70° C.

It is possible to use, as bifunctional derivative H—X-D-X—H, a branched polyester with a long alkyl chain and having at least two reactive groups, for example poly(12-hydroxystearate) with hydroxyl end groups. This polyester is obtained by auto-condensation of 1,2-hydroxystearic acid on itself, then reaction with a polyol to consume the residual acid groups. This oligomer has the formula (XI):

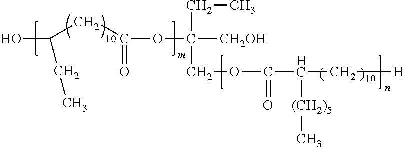

where the sum m+n is such that the oligomer has a number-average molecular weight of the order of 2000 and a hydroxyl functionality of the order of 1.8.

It is also possible to use, as bifunctional derivative H—X-D-X—H, natural or synthetic oils, bearing from two to three hydroxyl groups.

In a particular embodiment of the invention, oils bearing two hydroxyl groups per chain will be used, and preferably the monoglycerides of structure:

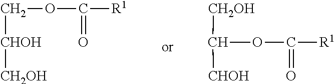

R¹ being a linear or branched C₈ to C₃₀ alkyl chain, for example glycerol monostearate.

These glycerol monoesters correspond for example to the bifunctional derivatives H—X-D-X—H, where:
D represents

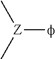

X represents —O—, and

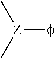

represents

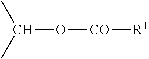

where R¹ is as defined previously.

When these glycerol monoesters are reacted with a diisocyanate, a solubilizing graft is introduced into the polymer chain, and not a block, as was the case with the bifunctional derivatives mentioned previously.

In a variant, a bifunctional derivative H—X-D-X—H will be used, selected from the oils bearing three hydroxyl groups per chain, for example castor oil, hydrogenated or not.

In this case, the reaction of polymerization is carried out with a shortage of diisocyanate relative to the stoichiometry of the reaction, to avoid crosslinking of the polymer and ensure it retains good solubility.

It is also possible to use diols with a long aliphatic chain. Advantageously, the diols of structure HO-D-OH will be used, where D is a linear or branched alkyl chain, having from 8 to 40 carbon atoms. These diols are marketed by the company ATOCHEM under the name VIKINOL®. We may also mention 1,12-dodecane diol and 1,10-decane diol, the latter being marketed by the company COGNIS under the trade name SOVERMOL 110®.

It is also possible to use the diols of structure

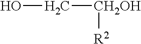

where R² is an alkyl chain, having from 8 to 40 carbon atoms.

These diols with a long aliphatic chain are, preferably, used with one or other of the derivatives H—X-D-X—H previously mentioned, to serve as chain couplers during the synthesis of polyurethanes and/or polyureas.

Finally, dimer diamines or diamines with a long aliphatic chain can be used as bifunctional derivative H—X-D-X—H.

Use of these reagents in the reaction of polymerization makes it possible to introduce urea groups instead of urethane groups into the polymer.

According to a particular embodiment of the invention, dimer diamines will be used having the same structure as the dimer diols described previously, i.e. dimer diamines bearing two primary amine functions instead of the hydroxyl groups.

These dimer diamines can be obtained from the transformation of dimeric fatty acids, such as dimer diols.

In a variant, diamines of structure H₂N-D-NH₂ can be used, where D is a linear or branched alkyl chain, having from 8 to 40 carbon atoms. These diamines are preferably used mixed with a bifunctional derivative H—X-D-X—H selected from the dimer diols and their derivatives, the polydienes and polyolefins with hydroxyl end groups, branched polyesters with a long alkyl chain, and the oils bearing from 2 to 3 hydroxyl groups, mentioned previously.

Among these diamines, we may mention:
1,10-diamino decane and 1,12-diamino dodecane, and the following diaminated oils marketed by the company AKZO NOBEL: cocopropylene diamine (distilled or not) DUOMEEN® C or CD, hydrogenated tallowpropylene diamine DUOMEEN® HT, C16-22 alkylpropylene diamine DUOMEEN® M, oleylpropylene diamine DUOMEEN® 0, tallowpropylene diamine DUOMEEN® T.

Regarding the monofunctional derivatives A1-XH and A2-XH, they are advantageously selected from the monohydric alcohols or monoamines with linear or branched alkyl chains having from 1 to 80 carbon atoms, the natural or synthetic oils bearing a single hydroxyl group per chain, for example the diesters of glycerols or the triesters of citric acid and fatty alcohol.

The polycondensation reactions envisaged are carried out conventionally in an organic solvent, able to dissolve the reactants and the polymer that forms. This solvent is preferably easily removable at the end of the reaction, notably by distillation and does not react with the isocyanate groups.

Generally, each of the reactants is dissolved in some of the organic solvent before the polymerization reaction.

It is sometimes desirable to use a catalyst for activating the polymerization. This will generally be selected from the catalysts commonly used in polyurethane and polyurea chemistry, for example tin 2-ethyl hexanoate.

The molar proportions of the principal reactants of the polymerization reaction depend on the chemical structure and molecular weight of the polymers (polyurethanes and/or polyureas) that we wish to obtain, as is conventionally the case in the chemistry of the polyurethanes and polyureas. Moreover, the order of introduction of the reactants will be adapted to said chemistry.

Thus, reaction of two moles of functional derivative H—X-D-X—H with one mole of diisocyanate gives, after the reactants have been consumed completely, a polymer defined by the formula (XII):

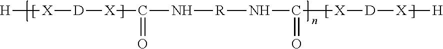

The procedure for this reaction is advantageously as follows:
the initial medium is a solution comprising two moles of derivative H—X-D-X—H, for example two moles of dimer diol, in a solvent, for example tetrahydrofuran,
a solution comprising one mole of diisocyanate dissolved in the same solvent, for example toluene diisocyanate dissolved in tetrahydrofuran, is added dropwise to this initial solution.

Moreover, equimolar reaction of a bifunctional derivative H—X-D-X—H with a diisocyanate, with consumption of the residual isocyanates by a monofunctional compound A₁-XH, gives a polymer defined by formula (XIII)

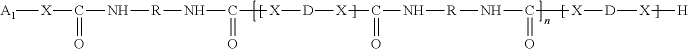

This reaction will then preferably be carried out by simultaneous addition, in a reactor, of an organic solution of one mole of H—X-D-X—H, for example a POLYTAIL® described previously, and of an organic solution of one mole of diisocyanate, for example 4, 4′-dicyclohexylmethane diisocyanate. Simultaneous addition of these two organic solutions is also called “double pouring”. At the end of double pouring, the reaction mixture is heated at 60° C. for 5 hours. Then a sample is taken from the reaction mixture to determine the residual isocyanates using a method known by a person skilled in the art. Finally, a solution of a selected monofunctional compound A1-X—H is added to the reaction mixture in a sufficient amount to consume the residual isocyanates, said amount having been estimated from the determination of the residual isocyanates. Advantageously, 1-decanol will be used as monofunctional derivative A1-X—H.

Finally, the reaction between
one mole of compound H—X-D-X—H, for example a dimer diol,
three moles of diisocyanate, for example 4,4′-dicyclohexyl methane diisocyanate, and
two moles of coupler of structure

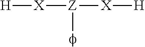

where φ is a linear, branched or cyclic aliphatic chain having from 8 to 20 carbon atoms, leads to the formation of a block and draft polymer of structure:

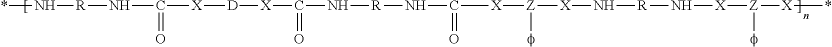

Any residual isocyanates can be consumed by adding a suitable amount of monofunctional reagent A1-X—H.

To obtain said polymer, the following procedure is used:
the initial reaction mixture is constituted of a solution comprising one mole of a bifunctional derivative H—X-D-X—H,
a solution of three moles of diisocyanate is added dropwise to this mixture,
it is then left to react for 3 hours at 60° C.:
then an organic solution comprising two moles of a coupler, defined by the formula

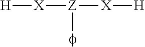

is poured into this mixture, and any residual isocyanates can be consumed by adding a suitable amount of monofunctional reagent A1-XH.

(9) Lipophilic Silicone Polymers:

The lipophilic polymeric silicone structure-forming agents are for example polymers of the polyorganosiloxane type such as those described in documents U.S. Pat. No. 5,874,069, U.S. Pat. No. 5,919,441, U.S. Pat. No. 6,051,216 and U.S. Pat. No. 5,981,680. According to the invention, the polymers used as structure-forming agent can belong to the following two families:

1) polyorganosiloxanes having at least two groups capable of establishing hydrogen interactions, these two groups being located in the chain of the polymer, and/or
2) polyorganosiloxanes having at least two groups capable of establishing hydrogen interactions, these two groups being located on grafts or branchings.

The groups capable of establishing hydrogen interactions can be selected from the ester, amide, sulphonamide, carbamate, thiocarbamate, urea, urethane, thiourea, oxamido, guanidino, and biguanidino groups and combinations thereof.

According to a first variant, the silicone polymers are polyorganosiloxanes as defined above, whose units capable of establishing hydrogen interactions are located in the chain of the polymer.

The silicone polymers can more particularly be polymers comprising at least one unit corresponding to general formula (XIV):

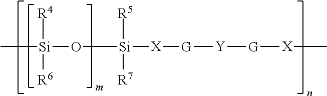

in which:
R⁴, R⁵, R⁶ and R⁷, which may be identical or different, represent a group selected from:
linear, branched or cyclic, C₁ to C₄₀ hydrocarbon groups, saturated or unsaturated, which can contain one or more oxygen, sulphur and/or nitrogen atoms in their chain, and which can be substituted partly or fully with fluorine atoms,
C₆ to C₁₀ aryl groups, optionally substituted with one or more C₁ to C₄ alkyl groups,
polyorganosiloxane chains, which may or may not contain one or more oxygen, sulphur and/or nitrogen atoms,
the X, which may be identical or different, represent a linear or branched C₁ to C₃₀ alkylene diyl group, which can contain one or more oxygen and/or nitrogen atoms in its chain,
Y is a linear or branched alkylene, arylene, cycloalkylene, alkylarylene or arylalkylene divalent group, saturated or unsaturated, of C₁ to C₅₀, which can have one or more oxygen, sulphur and/or nitrogen atoms, and/or bear as substituent one of the following atoms or groups of atoms: fluorine, hydroxy, C₃ to C₈ cycloalkyl, C₁ to C₄₀ alkyl, C₅ to C₁₀ aryl, phenyl optionally substituted with 1 to 3 C₁ to C₃ alkyl, C₁ to C₃ hydroxyalkyl and C₁ to C₆ amino alkyl groups, or
Y represents a group corresponding to the formula:

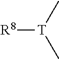

in which
T represents a trivalent or tetravalent, linear or branched, saturated or unsaturated, C₃ to C₂₄ hydrocarbon group, optionally substituted with a polyorganosiloxane chain, and can contain one or more atoms selected from O, N and S, or T represents a trivalent atom selected from N, P and Al, and
R⁸ represents a linear or branched C₁ to C₅₀ alkyl group, or a polyorganosiloxane chain, which can have one or more ester, amide, urethane, thiocarbamate, urea, thiourea and/or sulphonamide groups, which may or may not be bound to another chain of the polymer, the G, which may be identical or different, represent the divalent groups selected from:

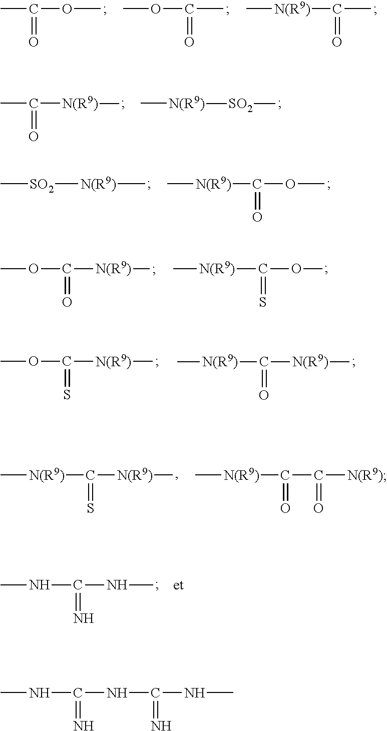

where R⁹ represents a hydrogen atom or a linear or branched, C₁ to C₂₀ alkyl group, provided that at least 50% of the R⁹ of the polymer represents a hydrogen atom and that at least two of the groups G of the polymer are a group other than:

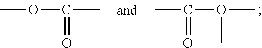

n is an integer in the range from 2 to 500, preferably from 2 to 200, and m is an integer in the range from 1 to 1000, preferably from 1 to 700 and more preferably from 6 to 200.

According to the invention, 80% of the R⁴, R⁵, R⁶ and R⁷ of the polymer are preferably selected from the methyl, ethyl, phenyl and 3,3,3-trifluoropropyl groups.

According to the invention, Y can represent various divalent groups, optionally additionally having one or two free valences for establishing bonds with other units of the polymer or copolymer. Preferably, Y represents a group selected from:
linear C₁ to C₂₀ alkylene groups, preferably C₁ to C₁₀,
branched alkylene groups which can have rings and unconjugated unsaturations, of C₃₀ to C₅₆,
C₅-C₆ cycloalkylene groups,
phenylene groups optionally substituted with one or more C₁ to C₄₀ alkyl groups,
C₁ to C₂₀ alkylene groups, having from 1 to 5 amide groups,
C₁ to C₂₀ alkylene groups, having one or more substituents, selected from hydroxyl groups, C₃ to C₈ cycloalkane, C₁ to C₃ hydroxyalkyl and C₁ to C₆ alkylamines,
polyorganosiloxane chains of formula:

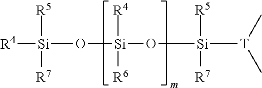

in which R⁴, R⁵, R⁶, R⁷, T and m are as defined above, and
polyorganosiloxane chains of formula:

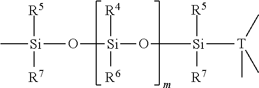

According to the second variant, the polyorganosiloxanes can be polymers comprising at least one unit corresponding to formula (XV):

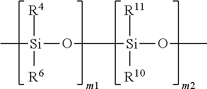

in which
R⁴ and R⁶, which may be identical or different, are as defined above for formula (XIV),
R¹⁰ represents a group as defined above for R⁴ and R⁶, or represents the group of formula —X-G-R¹² in which X and G are as defined above for formula (XIV) and R¹² represents a hydrogen atom or a linear, branched or cyclic, saturated or unsaturated, C₁ to C₅₀ hydrocarbon group, optionally having one or more atoms selected from O, S and N in its chain, optionally substituted with one or more fluorine atoms and/or one or more hydroxyl groups, or a phenyl group optionally substituted with one or more C₁ to C₄ alkyl groups,
R¹¹ represents the group of formula —X-G-R¹² in which X, G and R¹² are as defined above,
m₁ is an integer in the range from 1 to 998, and
m₂ is an integer in the range from 2 to 500.

According to the invention, the silicone polymer used as structure-forming agent can be a homopolymer, i.e. a polymer having several identical units, in particular units of formula (XIV) or of formula (XV).

According to the invention, we can also use a silicone polymer constituted of a copolymer having several different units of formula (XIV), i.e. a polymer in which at least one of the R⁴, R⁵, R⁶, R⁷, X, G, Y, m and n is different in one of the units. The copolymer can also be formed from several units of formula (XV), in which at least one of the R₄, R₆, R¹⁰, R¹¹, m₁ and m₂ is different in at least one of the units.

It is also possible to use a polymer having at least one unit of formula (XIV) and at least one unit of formula (XV), moreover the units of formula (XIV) and the units of formula (XV) may be identical to or different from one another.

According to a variant of the invention, it is also possible to use a polymer additionally comprising at least one hydrocarbon unit having two groups capable of establishing hydrogen interactions selected from the ester, amide, sulphonamide, carbamate, thiocarbamate, urea, urethane, thiourea, oxamido, guanidino, and biguanidino groups and combinations thereof.

These copolymers can be block copolymers, sequence copolymers or graft polymers.

According to an advantageous embodiment of the invention, the groups capable of establishing hydrogen interactions are amide groups of formula —C(O)NH— and —HN—C(O)—. In this case, the structure-forming agent can be a polymer comprising at least one unit of the following formula (XVI) or (XVII):

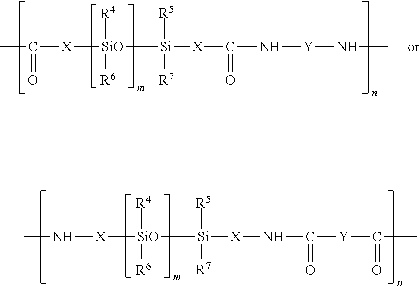

in which R⁴, R⁵, R⁶, R⁷, X, Y, m and n are as defined above.

Said unit can be obtained:
either by a condensation reaction between a silicone with α,ω-carboxylic acids end groups and one or more diamines, according to the following reaction scheme:

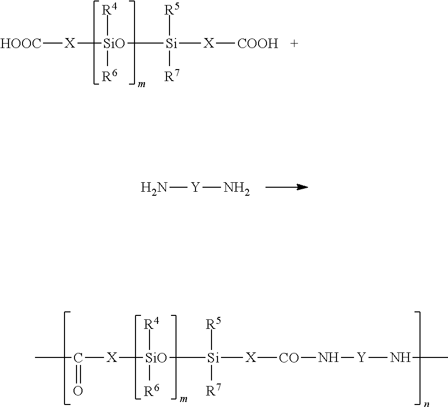

or by reaction of two molecules of α-unsaturated carboxylic acid with a diamine according to the following reaction scheme:

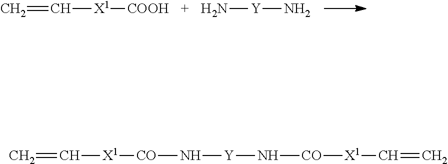

followed by the addition of a siloxane to the ethylenic unsaturations, according to the following scheme:

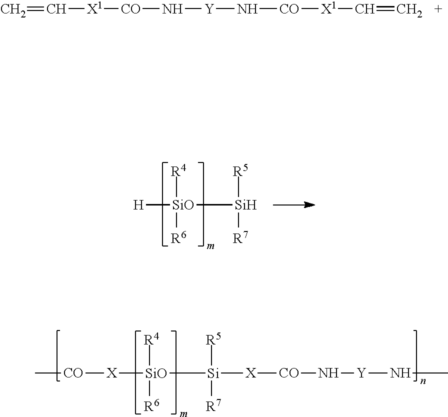

in which X¹—(CH₂)₂— corresponds to X defined above and Y, R⁴, R⁵, R⁶, R⁷ and m are as defined above,
or by reaction of a silicone with α,ω-NH₂ end groups and a diacid of formula HOOC—Y—COOH according to the following reaction scheme:

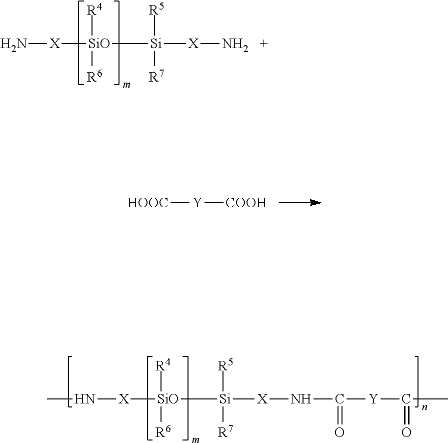

In these polyamides of formula (XVI) or (XVII), m is in the range from 1 to 700, in particular from 15 to 500 and notably from 50 to 200 and n is in particular from 1 to 500, preferably from 1 to 100 and more preferably from 4 to 25,

X is preferably a linear or branched alkylene chain having from 1 to 30 carbon atoms, in particular 1 to 20 carbon atoms, notably from 5 to 15 carbon atoms and more particularly 10 carbon atoms, and
Y is preferably an alkylene chain that is linear or branched or that can comprise rings and/or unsaturations, having from 1 to 40 carbon atoms, in particular from 1 to 20 carbon atoms, and more preferably from 2 to 6 carbon atoms, in particular 6 carbon atoms.

In formulae (XVI) and (XVII), the alkylene group representing X or Y can optionally contain, in its part alkylene, at least one of the following constituents:

1 to 5 amide, urea, urethane, or carbamate groups, a C₅ or C₆ cycloalkyl group, and
a phenylene group optionally substituted with 1 to 3 identical or different C₁ to C₃ alkyl groups.

In formulae (XVI) and (XVII), the alkylene groups can also be substituted with at least one constituent selected from the group comprising:

a hydroxyl group,
a C₃ to C₈ cycloalkyl group, one to three C₁ to C₄₀ alkyl groups,
a phenyl group optionally substituted with one to three C₁ to C₃ alkyl groups,
a C₁ to C₃ hydroxyalkyl group, and
a C₁ to C₆ aminoalkyl group.

In formulae (XVI) and (XVII), Y can also represent:

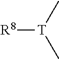

where R⁸ represents a polyorganosiloxane chain, and T represents a group of formula:

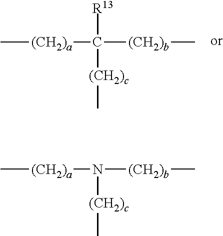

in which a, b and c are, independently, integers in the range from 1 to 10, and R¹³ is a hydrogen atom or a group such as those defined for R⁴, R⁵, R⁶ and R⁷.

In formulae (XVI) and (XVII), R⁴, R⁵, R⁶ and R⁷ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As already seen, the polymer can comprise units of formula (XVI) or (XVII), which may be identical or different.

Thus, the polymer can be a polyamide containing several units of formula (XVI) or (XVII) of different lengths, i.e. a polyamide corresponding to formula (XVIII):

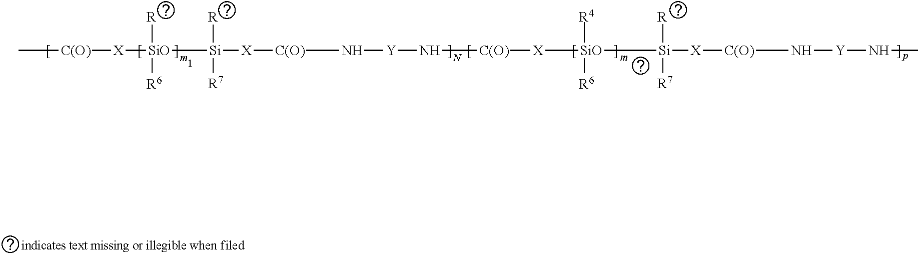

in which X, Y, n, R⁴ to R⁷ have the meanings given above, m₁ and m₂, which are different, are selected in the range from 1 to 1000, and p is an integer in the range from 2 to 300.

In this formula, the units can be structured to form either a block copolymer, or a random copolymer, or an alternating copolymer. In this copolymer, the units can have not only different lengths but also different chemical structures, for example can have different Y. In this case, the polymer can correspond to formula (XIX):

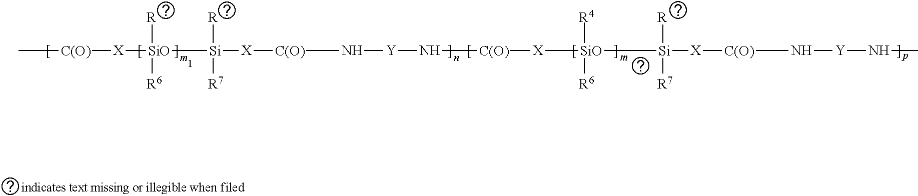

in which R⁴ to R⁷, X, Y, m₁, m₂, n and p have the meanings given above and Y¹ is different from Y but selected from the groups defined for Y. As previously, the various units can be structured to form either a block copolymer, or a random copolymer, or an alternating copolymer.

In this first embodiment of the invention, the structure-forming agent can also be constituted of a graft copolymer. Thus, the polyamide with silicone units can be grafted and optionally crosslinked by silicone chains with amide groups. Said polymers can be synthesized with trifunctional amines.

In this case, the polymer can comprise at least one unit of formula (XX):

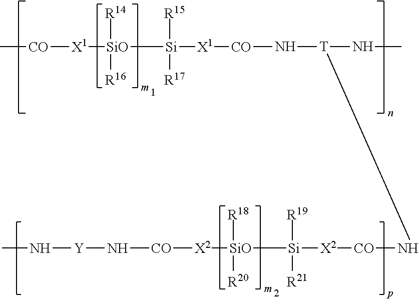

in which X² and X², which may be identical or different, have the meaning given for X in formula (XIV), n is as defined in formula (XIV), Y and T are as defined in formula (XIVI), R¹⁴ to R²¹ are groups selected from the same group as R⁴ to R⁷, m₁ and m₂ are numbers in the range from 1 to 1000, and p is an integer in the range from 2 to 500.

In formula (XX), it is preferable that:

p is in the range from 1 to 25, more preferably from 1 to 7,
R¹⁴ to R²¹ are methyl groups,
T corresponds to one of the following formulae:

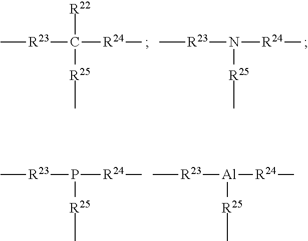

in which R²² is a hydrogen atom or a group selected from the groups defined for R⁴ to R⁷, and R²³, R²⁴ and R²⁵ are, independently, linear or branched alkylene groups, and more preferably to the formula:

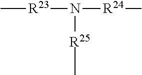

in particular with R²³, R²⁴ and R²⁵ representing —CH₂—CH₂—,
m₁ and m₂ are from 15 to 500, and more preferably from 15 to 45,
X¹ and X² represent —(CH₂)₁₀—, and
Y represents —CH₂—.

These polyamides with grafted silicone unit of formula (VII) can be copolymerized with silicone polyamides of formula (II) to form block copolymers, alternating copolymers or random copolymers. The percentage by weight of grafted silicone units (VII) in the copolymer can be from 0.5 to 30 wt. %.

According to the invention, as already seen, the siloxane units can be in the main chain or backbone of the polymer, but they can also be present in grafted or pendant chains. In the main chain, the siloxane units can be in the form of segments as described above. In the pendant or grafted chains, the siloxane units can appear individually or in segments.

According to one embodiment of the invention, a copolymer of silicone polyamide and of hydrocarbon polyamide can be used, i.e. a copolymer having units of formula (III) or (IV) and hydrocarbon polyamide units. In this case, the polyamide-silicone units can be located at the ends of the hydrocarbon polyamide.

Advantageously, the composition according to the invention comprises at least one polydimethylsiloxane block copolymer of general formula (I) having an index m with a value of about 15.

More preferably, the composition according to the invention comprises at least one polymer comprising at least one unit of formula (III) where m is in the range from 5 to 100, in particular from 10 to 75 and more particularly is of the order of 15; more preferably R⁴, R⁵, R⁶ and R⁷ represent, independently, a linear or branched C₁ to C₄₀ alkyl group, preferably a CH₃, C₂H₅, n-C₃H₇ or isopropyl group in formula (XVI).

As examples of silicone polymer that can be used, we may mention one of the silicone polyamides obtained according to examples 1 to 3 of document U.S. Pat. No. 5,981,680.

According to one embodiment of the invention, the polymer is constituted of a homopolymer or copolymer having urethane or urea groups. These polymers are described in detail in application WO 2003/106614 published on Dec. 24, 2003.

As previously, said polymer can comprise polyorganosiloxane units containing two or more urethane and/or urea groups, either in the backbone of the polymer, or on side chains or as pendant groups. The polymers having at least two urethane and/or urea groups in the backbone can be polymers comprising at least one unit corresponding to the following formula (XXI):

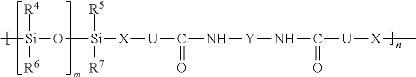

in which R⁴, R⁵, R⁶, R⁷, X, Y, m and n have the meanings given above for formula (XIV), and U represents —O— or —NH—, so that:

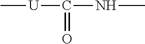

corresponds to a urethane or urea group.

In this formula (XXI), Y can be a linear or branched C₁ to C₄₀ alkylene group, optionally substituted with a C₁ to C₁₅ alkyl group or a C₅ to C₁₀ aryl group. Preferably, a —(CH₂)₆— group is used.

Y can also represent a C₅ to C₁₂ cycloaliphatic or aromatic group, which can be substituted with a C₁ to C₁₅ alkyl group or a C₅ to C₁₀ aryl group, for example a radical selected from the methylene-4-4-biscyclohexyl radical, the radical derived from isophorone diisocyanate, 2,4- and 2,6-tolylenes, 1,5-naphthylene, p-phenylene and 4,4′-biphenylene methane. Generally, it is preferred that Y represents a linear or branched C₁ to C₄₀ alkylene radical, or a C₄ to C₁₂ cycloalkylene radical.

Y can also represent a polyurethane or polyurea block corresponding to the condensation of several molecules of diisocyanate with one or more molecules of couplers of the diol or diamine type. In this case, Y has several urethane or urea groups in the alkylene chain. It can correspond to formula (XXII):

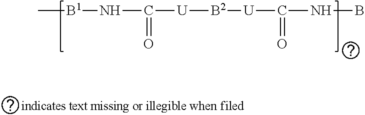

in which B¹ is a group selected from the groups given above for Y, U is —O— or —NH—, and B² is selected from:
the linear or branched C₁ to C₄₀ alkylene groups,
the C₅ to C₁₂ cycloalkylene groups, optionally bearing alkyl substituents, for example one to three methyl or ethyl, or alkylene groups, for example the radical of the diol: cyclohexane dimethanol, the phenylene groups optionally bearing C₁ to C₃ alkyl substituents, and
the groups of formula:

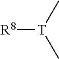

in which T is a trivalent hydrocarbon radical, which can contain one or more heteroatoms such as oxygen, sulphur and nitrogen and R⁸ is a polyorganosiloxane chain or a linear or branched C₁ to C₅₀ alkyl chain.

T can represent for example:

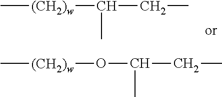

with w being an integer in the range from 1 to 10 and R⁸ being a polyorganosiloxane chain.

When Y is a linear or branched C₁ to C₄₀ alkylene group, the groups —(CH₂)₂— and —(CH₂)₆— are preferred.

In the formula given above for Y, d can be an integer in the range from 0 to 5, preferably from 0 to 3, more preferably equal to 1 or 2.

Preferably B² is a linear or branched C₁ to C₄₀ alkylene group, in particular —(CH₂)₂— or —(CH₂)₆—, or the group:

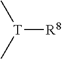

where R⁸ is a polyorganosiloxane chain.

As previously, the polymer constituting the texture-forming copolymer can be formed from silicone-urethane and/or silicone-urea units of different length and/or constitution, and can be in the form of block, sequenced or random copolymers.

The polymers of formula (XXII) having urea or urethane groups in the chain of the silicone polymer can be obtained by reaction between a silicone with α,ω-NH₂ or —OH end groups, of formula:

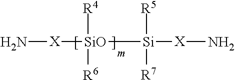

in which m, R⁴, R⁵, R⁶, R⁷ and X are as defined for formula (XIV), and a diisocyanate OCN—Y—NCO where Y has the meaning given in formula (XIV); and optionally a diol or diamine coupler of formula H₂N—B²—NH₂ or HO—B²—OH, where B₂ is as defined in formula (XXII).

According to the stoichiometric proportions between the two reactants, diisocyanate and coupler, Y can have formula (XXII) with d equal to 0 or d equal to 1 to 5.

As in the case of the silicone polyamides of formula (XVII), (XV) or (XVI), silicone polyurethanes or polyureas having units of different length and structure, in particular units with lengths differing by the number of silicone units, can be used in the invention. In this case, the copolymer can correspond for example to the formula:

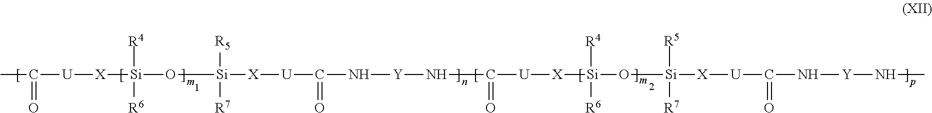

in which R⁴, R⁵, R⁶, R⁷, X, Y and U are as defined for formula (XXI) and m₁, m₂, n and p are as defined for formula (XVIII).

According to the invention, the silicone can also have urethane and/or urea groups, not in the backbone but in side branchings. In this case, the polymer can comprise at least one unit of formula (XXIV):

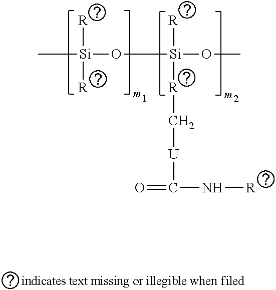

in which R⁴, R⁶, R⁵, m₁ and m₂ have the meanings given above for formula (XV), and R⁵ for formula (XIV),
U represents O or NH,
R²⁶ represents a C₁ to C₄₀ alkylene group, optionally having one or more heteroatoms selected from O and N, or a phenylene group, and
R²⁷ is selected from the linear, branched or cyclic, saturated or unsaturated C₁ to C₅₀ alkyl groups, and the phenyl groups optionally substituted with one to three C₁ to C₃ alkyl groups.

The polymers having at least one unit of formula (XXIV) contain siloxane units and urea or urethane groups, and they can be used as texture-forming copolymer in the compositions of the invention.

The siloxane polymers can have a single urea or urethane group per branching or can have branchings with two urea or urethane groups, or can contain a mixture of branchings with one urea or urethane group and branchings with two urea or urethane groups.

They can be obtained from branched polysiloxanes, having one or two amino groups per branching, by reacting these polysiloxanes with monoisocyanates.

As examples of starting polymers of this type having amino and diamino branchings, we may mention the polymers corresponding to the following formulae:

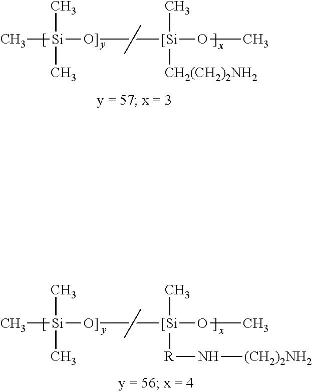

In these formulae, the symbol “/” indicates that the segments can be of different lengths and in a random order, and R represents a linear aliphatic group preferably having 1 to 6 carbon atoms and more preferably 1 to 3 carbon atoms.

Said branched polymers can be formed by reacting a siloxane polymer, having at least three amino groups per molecule of polymer, with a compound having a single monofunctional group (for example an acid, an isocyanate or isothiocyanate) so as to react this monofunctional group with one of the amino groups and form groups capable of establishing hydrogen interactions. The amino groups can be on side chains extending from the main chain of the siloxane polymer so that the groups capable of establishing hydrogen interactions are formed on these side chains, or else the amino groups can be at the ends of the main chain so that the groups capable of hydrogen interaction will be end groups of the polymer.

As the procedure for forming a polymer containing siloxane units and groups capable of establishing hydrogen interactions, we may mention the reaction of a siloxane diamine with a diisocyanate in a silicone solvent so as to supply a gel directly. The reaction can be performed in a silicone fluid, the resultant product being dissolved in the silicone fluid, at elevated temperature, the temperature of the system then being lowered in order to form the gel.

The preferred polymers for incorporation in the compositions according to the present invention are siloxane-urea copolymers, which are linear and contain urea groups as groups capable of establishing hydrogen interactions in the backbone of the polymer.

As an illustration of a polysiloxane terminated by four urea groups, we may mention the polymer of formula (XXV):

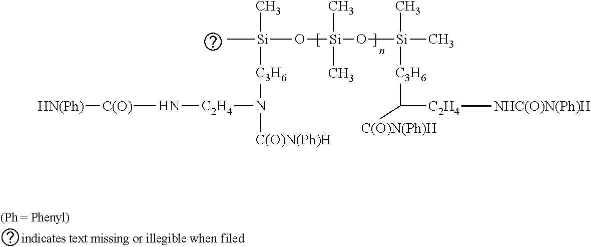

where Ph is a phenyl group and n is a number from 0 to 300, in particular from 0 to 100, for example 50.

This polymer is obtained by reaction of the following polysiloxane with amino groups:

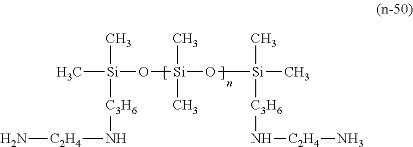

with phenyl isocyanate.

Branched silicone polyurethanes or polyureas can also be obtained using, instead of the diisocyanate OCN—Y—NCO, a triisocyanate of formula:

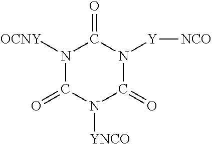

In this way a silicone polyurethane or polyurea is obtained having branchings bearing an organosiloxane chain with groups capable of establishing hydrogen interactions. Said polymer comprises for example a unit corresponding to formula (XXVI):

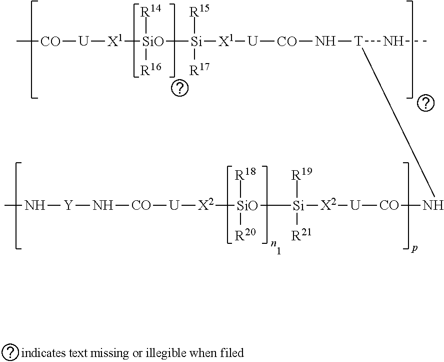

in which X¹ and X², which may be identical or different, have the meaning given for X in formula (XIV), n is as defined in formula (XIV), Y and T are as defined in formula (XIV), R¹⁴ to R²¹ are groups selected from the same group as the R⁴ to R⁷, m¹ and m² are numbers in the range from 1 to 1000, and p is an integer in the range from 2 to 500.

As in the case of the polyamides, in the invention it is possible to use copolymers of silicone polyurethane—or polyurea—and of hydrocarbon polyurethane or polyurea, carrying out the reaction of synthesis of the polymer in the presence of an α,ω-bifunctional block of non-silicone nature, for example a polyester, a polyether or a polyolefin.

As already seen, the copolymers of the invention can have siloxane units in the main chain of the polymer and groups capable of establishing hydrogen interactions, either in the main chain of the polymer or at the ends of the latter, or on side chains or branchings of the main chain. This can correspond to the following five arrangements:

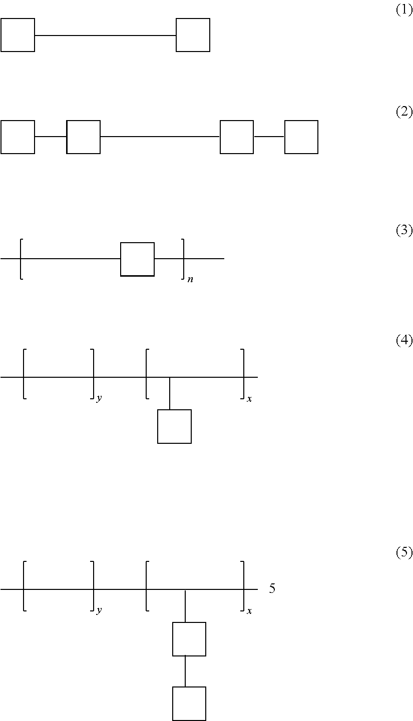

in which the continuous line is the main chain of the siloxane polymer and the squares represent the groups capable of establishing hydrogen interactions.

In case (1), the groups capable of establishing hydrogen interactions are positioned at the ends of the main chain. In case (2), two groups capable of establishing hydrogen interactions are arranged at each end of the main chain.

In case (3), the groups capable of establishing hydrogen interactions are arranged within the main chain in repeating units.

In cases (4) and (5), they are copolymers in which the groups capable of establishing hydrogen interactions are arranged on branchings of the main chain of a first series of units, which are copolymerized with units that do not have groups capable of establishing hydrogen interactions.

The polymers and copolymers used in the composition of the invention advantageously have a temperature of solid-liquid transition from 45° C. to 190° C. Preferably, they have a temperature of solid-liquid transition in the range from 70 to 130° C. and more preferably from 80° C. to 105° C.

(10) Organic Gelling Agents:

The oily structure-forming agent can also be selected from the non-polymeric molecular organic gelling agents, also called organic gelling agents, which are compounds whose molecules are capable of establishing physical interactions between them leading to self-aggregation of the molecules with formation of a supramolecular 3D network, which is responsible for the gelation of the oil(s) (also called liquid oily phase).

The supramolecular network can result from the formation of a network of fibrils (due to the stacking or aggregation of molecules of organic gelling agent), immobilizing the molecules of the liquid oily phase.

The ability to form this network of fibrils, and therefore to gel, depends on the nature (or chemical class) of the organic gelling agent, the nature of the substituents carried by its molecules for a given chemical class and the nature of the liquid oily phase.

The physical interactions are various but exclude co-crystallization. These physical interactions are in particular interactions such as self-complementary hydrogen interactions, π interactions between unsaturated rings, bipolar interactions, coordination bonds with organometallic derivatives and combinations thereof. In general, each molecule of an organic gelling agent can establish several types of physical interactions with a neighbouring molecule. Also, advantageously, the molecules of the organic gelling agents according to the invention have at least one group capable of establishing hydrogen bonds and more preferably at least two groups capable of establishing hydrogen bonds, at least one aromatic ring and more preferably at least two aromatic rings, at least one or more bonds with an ethylenic unsaturation and/or at least one or more asymmetric carbons. Preferably, the groups capable of forming hydrogen bonds are selected from the hydroxyl, carbonyl, amine, carboxylic acid, amide, urea, and benzyl groups and combinations thereof.

The organic gelling agent or agents according to the invention are soluble in the liquid oily phase after heating until a homogeneous transparent liquid phase is obtained. They can be solid or liquid at room temperature and atmospheric pressure.

The molecular organic gelling agent or agents usable in the composition according to the invention are notably those described in the document “Specialist Surfactants”, edited by D. Robb in 1997, p. 209-263, chapter 8 by P. Terech, in European applications EP-A-1068854 and EP-A-1086945 or in application WO-A-02/47031.

We may notably mention, among these organic gelling agents, the amides of carboxylic acids in particular tricarboxylic acids such as the cyclohexanetricarboxamides (see European patent application EP-A-1068854), the diamides having hydrocarbon chains each containing from 1 to 22 carbon atoms, for example from 6 to 18 carbon atoms, said chains being unsubstituted or substituted with at least one substituent selected from the ester, urea and fluoro groups (see application EP-A-1086945) and notably the diamides resulting from the reaction of diaminocyclohexane, in particular diaminocyclohexane in the trans form, and an acid chloride for example N,N′-bis(dodecanoyl)-1,2-diaminocyclohexane, the amides of N-acylamino acids such as the diamides resulting from the action of an N-acylamino acid with amines having from 1 to 22 carbon atoms, for example those described in document WO-93/23008 and notably the amides of N-acylglutamic acid where the acyl group represents a C₈ to C₂₂ alkyl chain such as dibutylamide of N-lauroyl-L-glutamic acid, manufactured or marketed by the company Ajinomoto under the name GP-1 and mixtures thereof.

It is also possible to use, as organic gelling agents, compounds of the bis-urea type of the following general formula:

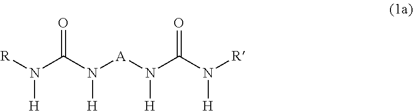

in which:
A is a group of formula (2a):

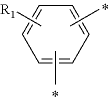

where R₁ is a linear or branched C₁ to C₄ alkyl radical, and the * symbolize the points of attachment of group A to each of the two nitrogen atoms of the residue of the compound of general formula (I), and
R and R′, which may be identical or different, are selected from:
i) the radicals of formula (3a):

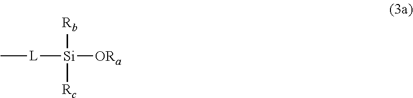

in which:
L is a single bond or a carbon-containing, notably hydrocarbon (alkylene) divalent radical, linear, branched and/or cyclic, saturated or unsaturated, comprising 1 to 18 carbon atoms, and which can comprise 1 to 4 heteroatoms selected from N, O and S;
Rₐ is:
   a) a carbon-containing, notably hydrocarbon (alkyl), linear, branched and/or cyclic, saturated or unsaturated radical, comprising 1 to 18 carbon atoms, and can comprise 1 to 8 heteroatoms selected from N, O, Si and S; or else
   b) a silicone radical of formula:

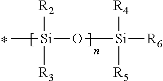

with n being between 0 and 100, notably between 1 and 80, or even 2 to 20;
and R₂ to R₆ are, independently of one another, carbon-containing, notably hydrocarbon (alkyl), linear or branched radicals, having 1 to 12, notably 1 to 6 carbon atoms, and can comprise 1 to 4 heteroatoms, notably O;
R_{b} and R_{c} are, independently of one another, selected from:
   a) carbon-containing, notably hydrocarbon (alkyl), linear, branched and/or cyclic, saturated or unsaturated radicals, comprising 1 to 18 carbon atoms, which can comprise 1 to 4 heteroatoms selected from N, O, Si and S;
   b) the radicals of formula:

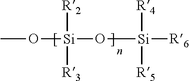

where n is between 0 and 100, notably between 1 and 80, or even 2 to 20;
and R′₂ to R′₆ are, independently of one another, carbon-containing, notably hydrocarbon (alkyl) linear or branched radicals, having 1 to 12, notably 1 to 6 carbon atoms, which can comprise 1 to 4 heteroatoms, notably O.
and
ii) linear, branched and/or cyclic, saturated or unsaturated C₁ to C₃₀ alkyl radicals, optionally comprising 1 to 3 heteroatoms selected from O, S, F and N;
   it being understood that at least one of the radicals R and/or R′ is of formula (3a).

Notably group A can be of formula:

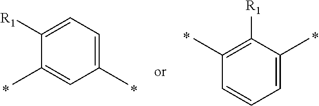

where R₁ and * are as defined previously.

In particular, R₁ can be a methyl group, which leads to a group A of formula:

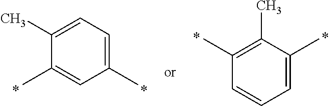

in which the * are as defined previously.

In particular, the compounds according to the invention can be in the form of a mixture in connection with the fact that A can be a mixture of 2,4-tolylene and 2,6-tolylene, notably in proportions (2,4 isomer)/(2,6 isomer) in the range from 95/5 to 80/20.

According to the invention, at least one of the radicals R and/or R′ must be of formula (3a):

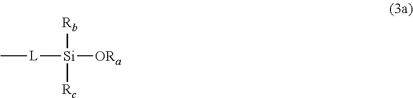

In this formula, L is preferably a carbon-containing, notably hydrocarbon (alkylene), linear, branched and/or cyclic, saturated or unsaturated divalent radical, comprising 1 to 18 carbon atoms, which can comprise 1 to 4 heteroatoms selected from N, O and S. In radical L, the carbon chain can be interrupted by the heteroatom(s) and/or can have a substituent comprising said heteroatom(s).

In particular, L can be of structure —(CH₂)ₙ— with n=1 to 18, notably 2 to 12, or even 3 to 8. Preferably, L is selected from the methylene, ethylene, propylene, butylene radicals and notably n-butylene or octylene.

The radical L can also be branched, for example of the type —CH₂—CH(CH₃)—, which leads to the radical of the following formula (3a):

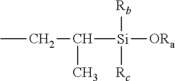

The radical Rₐ can be a carbon-containing radical, notably hydrocarbon (alkyl), linear, branched and/or cyclic, saturated or unsaturated, comprising 1 to 18 carbon atoms, and can comprise 1 to 8 heteroatoms selected from N, O, Si and S. The carbon chain can be interrupted by the heteroatom(s) and/or can comprise a substituent comprising said heteroatom(s); the heteroatoms can notably form one or more groups —SiO— (or —OSi)—.

Thus, the radical Ra can be of structure —(CH₂)n′-CH₃ with n′=0 to 17, notably 1 to 12, or even from 1 to 6. In particular, Ra can be methyl, ethyl, propyl or butyl.

It can also be of structure —(CH₂)ₓ—O—(CH₂)_{z}—CH₃ or else —(CH₂)ₓ—O—(CH₂)_{y}—O—(CH₂)_{z}—CH₃, with x=1 to 10, preferably 2; y=1 to 10 preferably 2, and z=0 to 10, preferably 0 or 1.

The radical Rₐ can moreover be of structure —SiR₄R₅R₆ (for the case where n=0), in which R₄, R₅ and R₆ are, independently of one another, preferably alkyl radicals, having 1 to 12 carbon atoms, notably 1 to 6 carbon atoms; in particular R₄, R₅ and/or R₆ can be selected from methyl, ethyl, propyl, butyl.

The radical Rₐ can also be a silicone radical of formula:

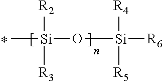

in which R₂ to R₆ are, independently of one another, preferably alkyl radicals, having 1 to 12 carbon atoms, notably 1 to 6 carbon atoms; in particular R₂ to R₆ can be selected from methyl, ethyl, propyl, butyl and in particular a radical:

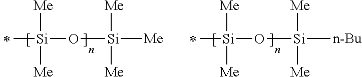

with n=1 to 100; and even more particularly a radical:

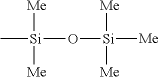

The radicals R_{b} and R_{c}, which may be identical or different, can be carbon-containing radicals, notably hydrocarbon (alkyl), linear, branched and/or cyclic, saturated or unsaturated, comprising 1 to 18 carbon atoms, and can comprise 1 to 8 heteroatoms selected from N, O, Si and S. In these radicals, the carbon chain can be interrupted by the heteroatom(s) and/or can comprise a substituent comprising said heteroatom(s); the heteroatoms can notably form one or more groups —SiO— (or —OSi)—.

Thus, they can be of structure —(CH₂)ₘ—CH₃ with m=0 to 17, notably 1 to 12, or even 2 to 5; in particular, R_{b} and/or R_{c} can be methyl, ethyl, propyl or butyl;

They can also be of structure —O—(CH₂)_{m′}—CH₃ with m′=0 to 5, notably 1 to 4, and in particular methoxy or ethoxy.

They can also be of structure —O— (CH₂)ₓ—O— (CH₂)z-CH₃ or —O—(CH₂)ₓ—O—(CH₂)_{y}—O—(CH₂)_{z}—CH₃, with x=1 to 10, preferably 2; y=1 to 10 preferably 2, and z=0 to 10, preferably 0 or 1.

They can also be of structure:

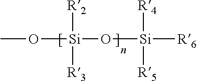

with n being between 0 and 100, notably between 1 and 80, or even 2 to 20;
and R′₂ to R′₆ are, independently of one another, preferably alkyl radicals, having 1 to 12 carbon atoms, notably 1 to 6 carbon atoms; in particular R′₂ to R′₆ can be selected from methyl, ethyl, propyl, butyl.

When they are of formula (3a), the radicals R and/or R′ are preferably selected from the following radicals

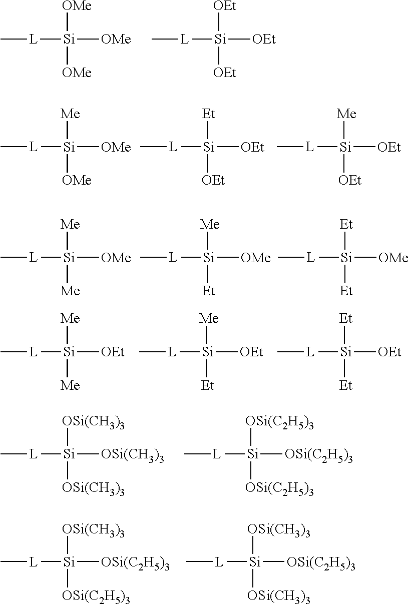

as well as those of formula:

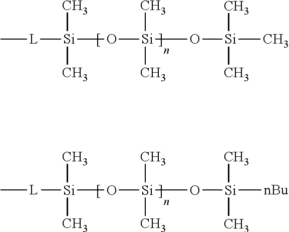

with n in the range from 0 to 100 and in particular

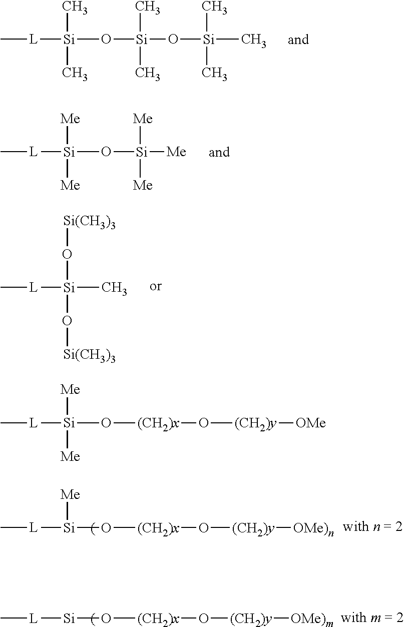

in which x=1 to 10, preferably 2; and y=1 to 10, preferably 2 and L is as defined above.

Preferably, in these formulae, L is a linear or branched C₁-C₈ alkylene radical, notably methylene, ethylene, propylene, butylene and notably n-butylene, octylene or of formula —CH₂—CH(CH₃)—.

In a particular embodiment, R and R′, which may be identical or different, are both of formula (3a).

In another embodiment, one of the radicals R or R′ represents a linear, branched and/or cyclic, saturated or unsaturated C₁ to C₃₀ alkyl radical, optionally comprising 1 to 3 heteroatoms selected from O, S, F and N.

This proves particularly advantageous for conferring a universal character on the compounds of formula (1a), i.e. allows them to be used as texture-forming agents for carbon-containing polar or nonpolar media, linear or cyclic silicone media, mixed oils i.e. carbon-containing partially siliconized oils, and mixtures thereof.

The carbon chain can be interrupted by the heteroatom(s) and/or can comprise a substituent comprising said heteroatom(s), notably in the form of a carbonyl group (—CO—), one or more hydroxyl radicals (—OH), and/or an ester radical —COOR″ with R″=linear or branched alkyl radical having 1 to 8 carbon atoms.

Thus, said radical R or R′ can be a group selected from:

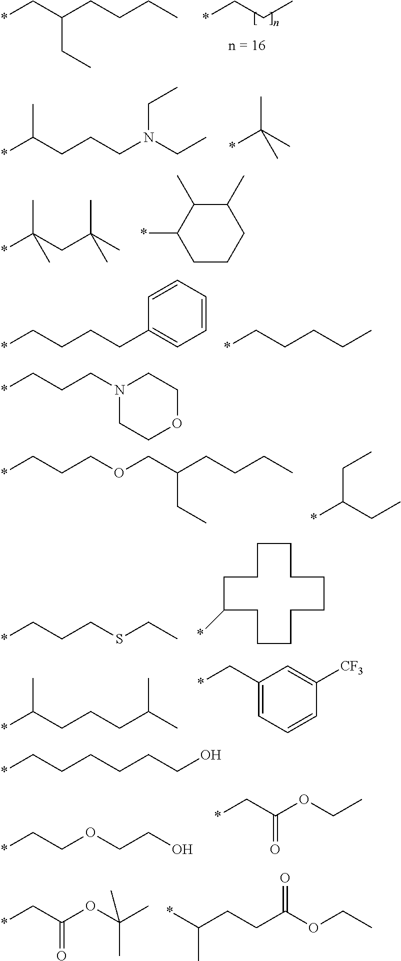

with * having the definition given above.

In a preferred embodiment, R or R′ represents a branched, notably monobranched, preferably non-cyclic, saturated or unsaturated alkyl radical, comprising 3 to 16 carbon atoms, notably 4 to 12, or even 4 to 8 carbon atoms, and optionally comprising 1 to 3 heteroatoms selected from O, S, F and/or N, preferably O and/or N.

In particular, R or R′ can be tert-butyl or 2-ethylhexyl radicals or of formula:

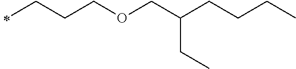

When the compound of formula (1a) comprises a radical R which is an alkyl radical, and therefore a radical R′ which is of formula (3a), the ratio of nR to nR′ is preferably between 5/95 and 95/5, for example between 10/90 and 90/10, in particular between 40/60 and 85/15, notably between 50/50 and 80/20, or even between 60/40 and 75/25;

where nR is the number of moles of amine NH₂—R and nR′ is the number of moles of amine NH₂—R′ used for preparing the compound of formula (1a).

The compounds according to the invention can be in the form of salts and/or isomers of compounds of formula (1a).

Generally, the compounds of general formula (I) according to the invention can be prepared as described in application FR2910809.

The compounds of the bis-urea silicone type described previously can be mixed with other non-silicone bis-urea compounds. The non-silicone bis-urea compounds can, according to a first aspect, correspond to the following general formula (Ib):

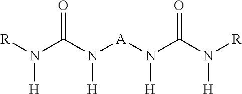

in which:
A is a group of formula:

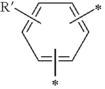

where R′ is a linear or branched C₁ to C₄ alkyl radical and the * symbolize the points of attachment of group A to each of the two nitrogen atoms of the residue of the compound of general formula (2), and
R is a, mono-branched, non-cyclic, saturated or unsaturated C₆ to C₁₅ alkyl radical whose hydrocarbon chain is optionally interrupted by 1 to 3 heteroatoms selected from O, S and N, or
   a salt or isomer thereof.

According to a preferred embodiment of the invention, the group represented by A is a group of formula:

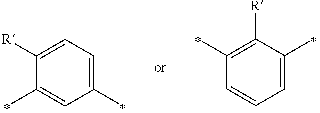

where R′ and * are as defined previously.

In particular, R′ can be a methyl group, and group A is then more particularly a group of formula:

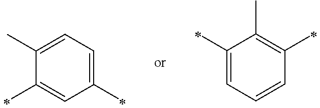

where * is as defined previously.

According to a first embodiment of the invention, R can be selected from the mono-branched radicals of general formula CₙH₂ₙ₊₁, n being an integer in the range from 6 to 15, in particular from 7 to 9 or even equal to 8.

Thus, the two groups R of the compound of formula (2) can represent respectively a group:

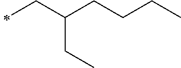

with * symbolizing the point of attachment of each of the groups R to each of the nitrogen atoms of the residue of the compound of general formula (2a).

According to a second embodiment of the invention, R can be selected from the mono-branched radicals of general formula Cm-pH2m+1−2pXp, p being equal to 1, 2 or 3, preferably equal to 1, m being an integer in the range from 6 to 15, preferably from 10 to 14, in particular from 10 to 12, or even equal to 11 and X representing sulphur and/or oxygen atoms, in particular oxygen atoms.

More particularly, R can be a radical of formula C_{m′}H2_{m′}X—(C_{p′}H_{2p′}X′)ᵣ—CxH₂ₓ₊₁, in which X and X′ are, independently of one another, an oxygen or sulphur atom, preferably oxygen, r is 0 or 1, m′, p′ and x are integers such that their sum is in the range from 6 to 15, in particular from 10 to 12, or is equal to 11 and it is understood that at least one of the carbon-containing chains C_{m′}H_{2m′}, C_{p′}H2_{p′}, or CₓH₂ₓ₊₁ is branched.

Preferably it is the chain CₓH₂ₓ₊₁ that is branched, preferably r=0, preferably m′ is an integer in the range from 1 to 10, notably from 2 to 6, in particular equal to 3, and/or preferably x is an integer in the range from 4 to 16, notably from 6 to 12, in particular equal to 8.

Thus, the two groups R of the compound of formula (1a) can represent respectively a group:

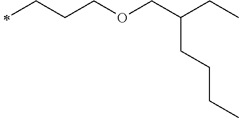

with * symbolizing the point of attachment of each of the groups R to each of the nitrogen atoms of the residue of the compound of general formula (1).

Such compounds can be present in the compositions according to the invention mixed with isomers, notably position isomers on group A, notably in proportions 95/5 or 80/20.

As can be seen from the examples given below, the presence of one or other of its radicals in the molecule of general formula (2) proves particularly advantageous for imparting a universal character, in the sense of the invention, to the corresponding non-silicone bis-urea derivatives.

As representative and non-limiting examples of compounds suitable quite particularly for the invention, we may more particularly mention the following compounds, used alone or mixed:

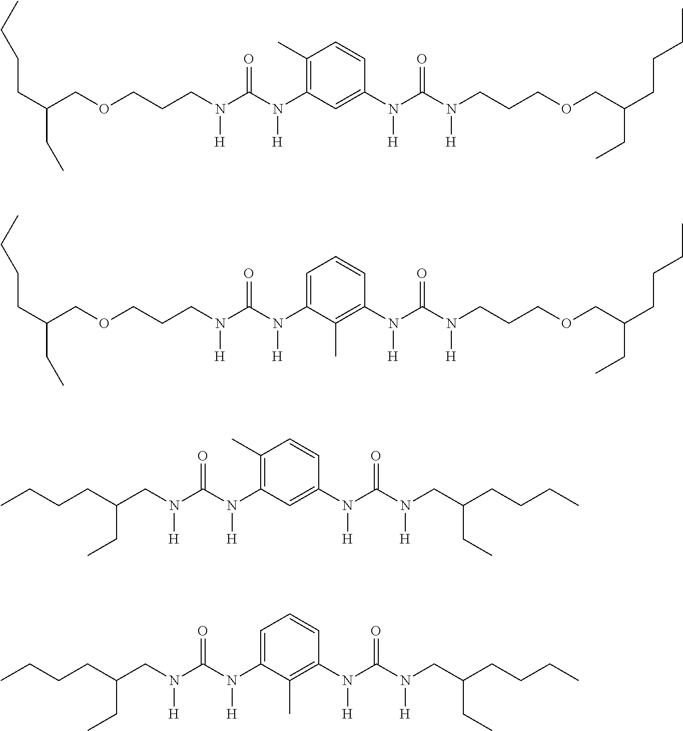

and their salts.

According to another aspect of the invention, the non-silicone bis-urea derivatives of the following formula (3a):

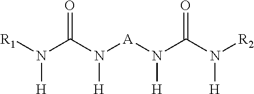

in which:
A is a group of formula:

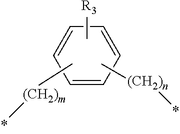

where
R₃ is a hydrogen atom or a linear or branched C₁ to C₄ alkyl radical,
n and m are, independently of one another, equal to 0 or 1, and
* symbolizes the point of attachment of group A to the two nitrogen atoms of the residue of the compound of general formula (3),
R₁ is a, branched, non-cyclic, saturated or unsaturated C₃ to C₁₅ carbon-containing radical, optionally containing from 1 to 3 heteroatoms selected from O, S, F and N and/or a carbonyl, and combinations thereof,
R₂ is different from R₁ and is selected from the saturated or unsaturated, linear, branched or cyclic C₁-C₂₄ alkyl radicals, optionally containing from 1 to heteroatoms selected from O, S, F and N, and optionally substituted with:
1, 2 or 3 hydroxyl radicals,
a ester radical (—COOR₄), where R₄ is a linear or branched alkyl radical, having from 1 to 8, notably 1 to 6, or even 2 to 4 carbon atoms;
a saturated, unsaturated or aromatic cyclic radical having from 5 to 12 carbon atoms, in particular a phenyl radical optionally substituted with one or more radicals, which may be identical or different, selected from the C₁-C₄ alkyl radicals, trifluoromethyl, or a morpholine derivative, and/or
one or more linear or branched C₁-C₄ alkyl radicals,
   or a salt or isomer thereof.

In particular n and m are equal, and more particularly equal to zero and R₃ is a radical R′₃, as defined below. Thus, preferably, A represents a group

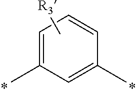

where R₃′ is a linear or branched C₁ to C₄ alkyl radical and * symbolizes the point of attachment of group A to the two nitrogen atoms of the residue of the compound of general formula (3).

According to a variant of the invention, the compound of general formula (3a) comprises, as A, at least one group selected from:

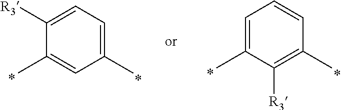

where R₃′ and * are as defined previously.

In particular, R₃′ can be a methyl group, and in this case group A represents a group

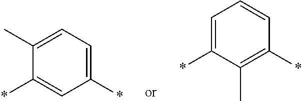

* being as defined previously.

In particular, the compounds are such that A is a mixture of 2,4-tolylene and 2,6-tolylene notably in proportions (2,4 isomer)/(2,6 isomer) in the range from 95/5 to 80/20.

According to one embodiment of the invention, the compound of general formula (3a) comprises, as R₁, a branched C₆-C₁₅ radical.

According to one embodiment of the invention, the compound of general formula (3a) comprises, as R₁, a group selected from:

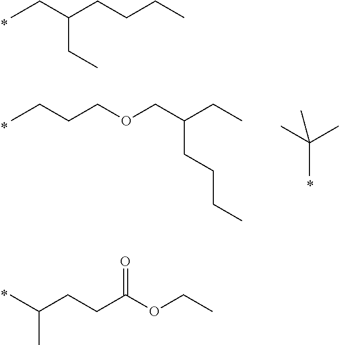

with * symbolizing the point of attachment of group R₁ to the nitrogen of the residue of the compound of general formula (3a).

As follows from the examples given below, the presence of one and/or other of its two radicals in the molecule of general formula (3a) proves particularly advantageous for imparting a universal character, in the sense of the invention, to the corresponding asymmetric bis-urea derivatives.

With regard to R₂, which is different from R₁, it can be selected advantageously from the following groups:

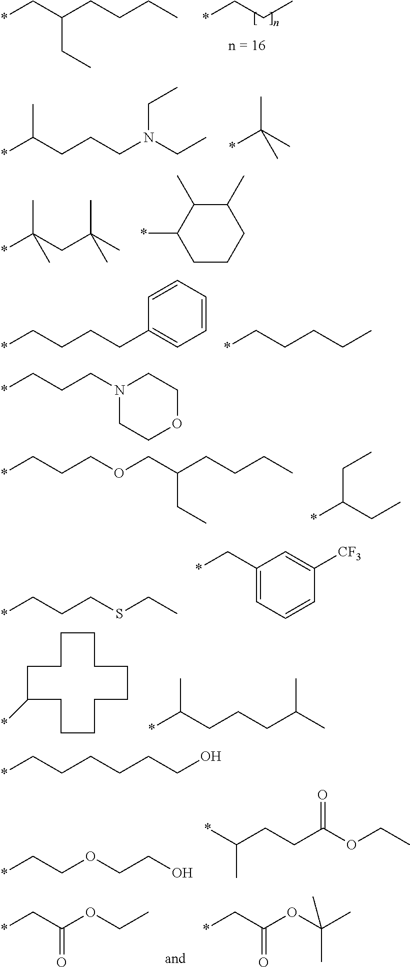

with * symbolizing the point of attachment of the group R₂ to the nitrogen of the residue of the compound of general formula (3a).

Generally, the compounds described can be prepared as described in application FR2910809.

(11) Block Copolymers:

It is also possible to use, as rheological agent of the fatty phase, graft or sequence block copolymers.

Graft or sequence block copolymers can notably be used, comprising at least one block of the polyorganosiloxane type and at least one block of a radical polymer, such as the graft copolymers of the acrylic/silicone type, which can notably be used when the non-aqueous medium is silicone.

It is also possible to use graft or sequence block copolymers comprising at least one block of the polyorganosiloxane type and at least a polyether. The polyorganopolysiloxane block can notably be a polydimethylsiloxane or alternatively a polyalkyl(C₂-C₁₈)methylsiloxane; the polyether block can be a C₂-C₁₈ polyalkylene, in particular polyoxyethylene and/or polyoxypropylene. In particular, the copolyol dimethicones or alkyl (C₂-C₁₈) copolyol dimethicones can be used, such as those sold under the name “Dow Corning 3225C” by the company Dow Corning, lauryl methicones such as those sold under the name “Dow Corning Q2-5200” by the company Dow Corning.

As graft or sequence block copolymers, we may also mention those comprising at least one block resulting from the polymerization of at least one ethylene monomer, with one or more optionally conjugated ethylenic bonds, such as ethylene or dienes such as butadiene and isoprene, and of at least one block of a vinyl and more preferably styrene polymer. When the ethylene monomer has several optionally conjugated ethylenic bonds, the residual ethylenic unsaturations after polymerization are generally hydrogenated. Thus, as is known, the polymerization of isoprene leads, after hydrogenation, to the formation of the ethylene-propylene block, and the polymerization of butadiene leads, after hydrogenation, to the formation of the ethylene-butylene block. Among these polymers, we may mention the block copolymers, notably of the “diblock” or “triblock” type such as polystyrene/polyisoprene (SI), polystyrene/polybutadiene (SB) such as those sold under the name ‘LUVITOL HSB’ by BASF, of the polystyrene/copoly(ethylene-propylene) type (SEB) such as those sold under the name ‘Kraton’ by Shell Chemical Co or of the polystyrene/copoly(ethylene-butylene) type (SEB). In particular, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS), Kraton D-1107 (SIS) can be used. The polymers are generally called hydrogenated or non-hydrogenated diene copolymers.

It is also possible to use Gelled Permethyl 99A-750, 99A-753-59 and 99A-753-58 (mixture of triblock and star-block copolymer), Versagel 5960 from Penreco (triblock+star-block copolymer); OS129880, OS129881 and OS84383 from Lubrizol (styrene/methacrylate copolymer).

As graft or sequence block copolymers comprising at least one block resulting from the polymerization of at least one ethylene monomer with one or more ethylenic bonds and at least one block of an acrylic polymer, we may mention the bi- or trisequence poly(methyl methylacrylate)/polyisobutylene copolymers or graft copolymers with a poly(methyl methylacrylate) backbone and polyisobutylene grafts.

As graft or sequence block copolymers comprising at least one block resulting from the polymerization of at least one ethylene monomer with one or more ethylenic bonds and at least one block of a polyether such as a C₂-C₁₈ polyalkylene (polyethylated and/or polypropoxylated notably), we may mention the bi- or trisequence polyoxyethylene/polybutadiene or polyoxyethylene/polyisobutylene copolymers.

(12) Silicone Elastomers as Gelling Agent of the Oily Phase

“Elastomer” means a flexible and deformable solid material, having viscoelastic properties and notably the consistency of a sponge. This elastomer is formed from polymer chains of high molecular weight whose mobility is limited by a uniform network of crosslinks.

The elastomeric organopolysiloxanes used in the composition according to the invention are preferably partially or fully crosslinked. They are in the form of particles. In particular, the particles of elastomeric organopolysiloxane have a size in the range from 0.1 to 500 μm, preferably from 3 to 200 μm and more preferably from 3 to 50 μm. These particles can have any shape and for example can be spherical, flat or amorphous.

When they are incorporated in an oily phase, these elastomeric organopolysiloxanes are transformed, depending on the proportion of oily phase used, to a product with a spongy appearance when they are used in the presence of low contents of oily phase, or to a homogeneous gel in the presence of larger amounts of oily phase. The gelation of the oily phase by these elastomers can be total or partial.

Thus, the elastomers of the invention can be carried in the form of anhydrous gel constituted of an elastomeric organopolysiloxane and an oily phase. The oily phase used during the manufacture of the anhydrous gel of elastomeric organopolysiloxane contains one or more oils that are liquid at ambient temperatures (25° C.) selected from the hydrocarbon oils and/or the silicone oils. Advantageously, the oily phase is a silicone liquid phase, containing one or more oils selected from the polydimethylsiloxanes with a linear or cyclic chain, which are liquid at room temperature, optionally having an alkyl or aryl chain, either pendant or at the end of the chain, the alkyl chain having from 1 to 6 carbon atoms.

According to one embodiment, the elastomeric organopolysiloxanes used according to the invention can be obtained by a reaction of addition and crosslinking, in the presence of a catalyst, preferably a catalyst of the platinum type, of at least:
(i) an organopolysiloxane having two vinylic groups in position α-ω of the silicone chain per molecule; and
(ii) an organopolysiloxane having at least two hydrogen atoms bound to one silicon atom per molecule.

The first organopolysiloxane (i) is selected from the polydimethylsiloxanes; it is preferably an α-ω-dimethylvinyl polydimethylsiloxane.

The organopolysiloxane is preferably in a gel obtained in the following stages:
(a) mixing of the first and second organopolysiloxanes (i) and (ii);
(b) addition of an oily phase to the mixture from stage (a);
(c) polymerization of the first and second organopolysiloxanes (i) and (ii) in an oily phase in the presence of a catalyst, preferably a platinum catalyst.

According to one embodiment, the crosslinked organopolysiloxane can be obtained by a polymeric addition reaction of an organohydrogenopolysiloxane of formula (4) with an organopolysiloxane of formula (5) and/or an unsaturated hydrocarbon chain of formula (6).

According to a variant, the crosslinked organopolysiloxane is obtained by a polymeric reaction of an organohydrogenopolysiloxane of formula (4) with an organopolysiloxane of formula (5).

### Organohydrogenopolysiloxane of Formula (4)

The organohydrogenopolysiloxane of formula (4) comprises at least one structural unit selected from the group comprising an SiO₂ unit, an HSiO_{1.5} unit, an RSiO_{1.5} unit, an RHSiO unit, an R₂SiO unit, an R₃SiO_{0.5} and an R₂HSiO_{0.5} unit, the group R in these units being a monovalent hydrocarbon chain having from 1 to 16 carbon atoms, which can be substituted or unsubstituted but is different from an unsaturated aliphatic group, and on average possesses at least 1.5 hydrogen atoms bound to a silicon atom.

The group R in the organohydrogenopolysiloxane of formula (4) can be an alkyl group having from 1 to 16, preferably from 10 to 16 carbon atoms. This group R can for example be a methyl group, an ethyl group, a propyl group, a lauryl group, a myristyl group and a palmityl group.

The group R in the organohydrogenopolysiloxane of formula (4) can also be an aryl group such as a phenyl or tolyl group.

The group R, still in the organohydrogenopolysiloxane of formula (4), can also be a monovalent hydrocarbon chain comprising a cycloalkyl group such as cyclohexyl or else a hydrocarbon chain substituted with one, two or more groups selected from a halogen atom such as chlorine, bromine, fluorine and a cyano group, for example an α-trifluoropropyl or chloromethyl group.

In particular, it is preferred that the group R represents at least 30 mol. % of methyl group and from to 50 mol. %, preferably from 10 to 40 mol. % of hydrocarbon chain having from 10 to 16 carbon atoms.

The hydrocarbon chain can then advantageously have at least one lauryl group, or even the majority of the groups R can be lauryl groups.

The organohydrogenopolysiloxane of formula (4) can be linear, branched or cyclic.

The organohydrogenopolysiloxane of formula (4) preferably contains from 2 to 50 and even more preferably 2 to 10 hydrogen atoms bound to a silicon atom (Si—H). The content of hydrogen atoms bound to a silicon atom in this compound of formula (I) varies conventionally from 0.5 to 50 mol. %, and even more preferably from 1 to 20 mol. % relative to the total sum of hydrogen atoms and of all the organic groups bound to a silicon atom.

### Organopolysiloxane of Formula (5)

The organopolysiloxane of formula (5) comprises at least one structural unit selected from the group comprising an SiO₂ unit, a (CH₂═CH)SiO1.5 unit, an RSiO_{1.5} unit, an R(CH₂═CH)SiO unit, an R₂SiO unit, an R₃SiO0.5 and an R₂(CH₂═CH)SiO_{0.5} unit, the group R being as defined in formula (I) and possessing on average at least 1.5 vinylic groups bound to a silicon atom.

This compound preferably contains from 2 to 50 vinylic groups bound to a silicon atom. The average number of vinylic groups bound to a silicon atom is preferably from 2 to 10, and even more preferably 2 to 5.

Preferably, at least 30 mol. % of the groups R are methyl groups and 5 to 50 mol. %, preferably 10 to 40 mol. % of the groups R are a hydrocarbon chain having from 10 to 16 carbon atoms.

The organopolysiloxane of formula (5) can be linear, branched or cyclic.

The content of vinylic groups in the compound of formula (5) is preferably between 0.5 and 50 mol. %, even more preferably 1 to 20 mol. % relative to all the organic groups bound to a silicon atom.

### Optional Unsaturated Hydrocarbon Chain of Formula (6)

The unsaturated hydrocarbon chain of formula (III) corresponds to the following formula:

CₘH₂ₘ₋₁(CH₂)ₓCₘH₂ₘ₋₁

in which
m is an integer in the range from 2 to 6 and
x is an integer equal to at least 1.
x is preferably an integer in the range from 1 to 20.

As examples of this compound of formula (6), we may mention pentadiene, hexadiene, heptadiene, octadiene, pentadecadiene, heptadecadiene and pentatriacontadiene.

The polymeric reactions of addition are described in detail in document US 2004/0234477.

Among the crosslinked organopolysiloxanes, crosslinked polyalkyl dimethylsiloxanes are preferred. Polyalkyl dimethylsiloxane means a linear organopolysiloxane of formula (7)

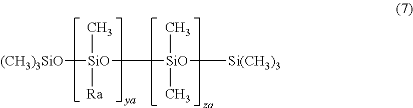

having grafts, bound monovalently or divalently, of formula (8)

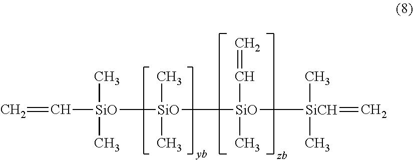

in which:
Ra is an alkyl group having from 10 to 16 carbon atoms, and can preferably be a lauryl group,
ya is an integer from 1 to 100;
za is an integer from 1 to 100,
yb is an integer from 1 to 100,
zb is an integer from 1 to 100.

“Bound divalently” means bound to two different organopolysiloxanes of formula (7). In other words it is a bridge between two linear chains as defined by formula (7).

The dimethicone/vinyldimethicone copolymers (INCI name: Dimethicone/Vinyldimethicone crosspolymer), and the vinyldimethicone/alkyl dimethicone copolymers, such as the vinyldimethicone/lauryl dimethicone copolymers (INCI name: Vinyl Dimethicone/Lauryl Dimethicone Crosspolymer) are preferably used as non-emulsifying elastomers usable according to the invention.

As non-emulsifying elastomers that can be used according to the invention, we may mention:
those with the INCI name Dimethicone/Vinyldimethicone Crosspolymer (and) C12-14 Pareth-12: such as those sold under the name “DC 9509” by the company Dow Corning,
those with the INCI name Dimethicone/Vinyldimethicone Crosspolymer: such as those sold under the name “DC9505”, or “DC 9506” by the company Dow Corning, those with the INCI name Cyclomethicone (and) Dimethicone/Vinyldimethicone Crosspolymer: such as those sold as KSG-15® by Shin-Etsu, Mel Trimethicone (and) Dimethicone/Vinyldimethicone Crosspolymer: such as those sold by Shin-Etsu as KSG-1610®,
those with the INCI name Dimethicone (and) Dimethicone/Vinyldimethicone Crosspolymer: such as those sold as KSG-16® by Shin-Etsu, Isododecane (and) Dimethicone/Vinyldimethicone Crosspolymer: such as those sold as USG-106® by Shin-Etsu,
those with the INCI name: Vinyl Dimethicone/Lauryl Dimethicone Crosspolymer: KSG-41® (in a mineral oil), KSG-42® (in isododecane), KSG-43® (in triethylhexanoine) and KSG-44® (in squalane), sold by Shin-Etsu.

As non-emulsifying elastomer, we may also mention spherical silicone non-emulsifying elastomers in the form of powder of crosslinked organopolysiloxane elastomer coated with silicone resin, notably silsesquioxane resin, as described for example in U.S. Pat. No. 5,538,793. These elastomers are sold under the names “KSP-100”, “KSP-101”, “KSP-102”, “KSP-103”, KSP-104″, “KSP-105” by the company Shin Etsu.

Other crosslinked elastomeric organopolysiloxanes in the form of spherical powders can be powders of hybrid silicone functionalized by fluoroalkyl groups, notably sold under the name “KSP-200” by the company Shin Etsu; powders of hybrid silicones functionalized by phenyl groups, notably sold under the name “KSP-300” by the company Shin Etsu.

It is also possible to use, in the compositions according to the invention, silicone elastomers with a group MQ, such as those sold by the company Wacker under the names Belsil RG100, Belsil RPG33 and preferably RG80. These particular elastomers, when used in combination with the resins according to the invention, can make it possible to improve the non-transfer properties of the compositions containing them.

(13) Cholesteric Liquid Crystal Agents:

The term “liquid crystal agents” means compounds that generate a mesomorphic state, i.e. a state where melting of the crystals gives rise to liquids possessing optical properties comparable to those of certain crystals. These compounds are defined more precisely in the chapter on Liquid Crystals in Ullmann's encyclopedia.

These liquid crystal agents are described in particular in the patents or patent applications EP 545 409, WO 94109086, EP 709 445, GB 2 282 145, GB 2 276 883, WO 95132247, WO 95132248, EP 686 674, EP 711 780.

These liquid crystal agents can react in response to vibrations by change in viscosity and/or by a colour change. More particularly, the compounds generating a mesomorphic state are compounds with a cholesteric function, whose structure is as follows:

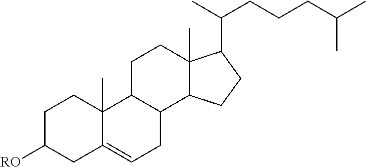

R is an alkyl, alkylcarbonyl group comprising 1 to 30 carbon atoms, unsubstituted or substituted with cyclic, aromatic groups, halogens, branched or unbranched.

As non-limiting examples, we may mention as liquid crystal agents corresponding to this definition: cholesterol erucyl carbonate, cholesterol methyl carbonate, cholesterol oleyl carbonate, cholesterol para-nonyl phenyl carbonate, cholesterol phenyl carbonate, cholesterol acetate, cholesterol benzoate, cholesterol butyrate, cholesterol isobutyrate, cholesterol chloride, cholesterol chloroacetate, cholesterol cinnamate, cholesterol crotanoate, cholesterol decanoate, cholesterol erucate, cholesterol heptanoate, cholesterol hexanoate, cholesterol myristate, cholesterol nonaoate, cholesterol octanoate, cholesterol oleate, cholesterol propionate, cholesterol valerate, dicholesteryl carbonate.

(14) Waxes:

Wax, in the sense of the present invention, means a lipophilic compound, solid at room temperature (25° C.), with reversible solid/liquid change of state, having a melting point greater than or equal to 30° C., which can be up to 120° C.

The melting point of a wax can be measured by means of a differential scanning calorimeter (DSC), for example the calorimeter sold under the name DSC 30 by the company METLER.

Waxes can be hydrocarbon-containing, fluorinated and/or siliconized and can be of vegetable, mineral, animal and/or synthetic origin. In particular, the waxes have a melting point above 25° C. and more preferably above 45° C.

Hydrocarbon waxes can notably be used, such as beeswax, lanolin wax, and Chinese insect waxes; rice wax, Carnauba wax, ouricury wax, alfa wax, cork fibre wax, sugar cane wax, Japan wax and sumach wax; montan wax, microcrystalline waxes, paraffins; polyethylene waxes, waxes obtained by Fischer-Tropsch synthesis and waxy copolymers as well as their esters.

We may also mention waxes obtained by catalytic hydrogenation of animal or vegetable oils having linear or branched C₈-C₃₂ fatty chains.

Among the latter, we may notably mention hydrogenated jojoba oil, hydrogenated sunflower oil, hydrogenated castor oil, hydrogenated copra oil and hydrogenated lanolin oil, and di-(trimethylol-1,1,1-propane) tetrastearate sold under the name “HEST 2T-4S” by the company HETERENE, di-(trimethylol-1,1,1-propane) tetrabehenate sold under the name HEST 2T-4B by the company HETERENE.

We may also mention the fluorinated waxes.

It is also possible to use the wax obtained by hydrogenation of oil olive esterified with stearyl alcohol sold under the name “PHYTOWAX Olive 18 L 57” or alternatively the waxes obtained by hydrogenation of castor oil esterified with cetyl alcohol sold under the name “PHYTOWAX ricin 16L64 et 22L73”, by the company SOPHIM. These waxes are described in application FR-A-2792190.

According to a particularly preferred embodiment of the invention, the following will be selected

(i) the polyolefin waxes resulting from the polymerization and notably homopolymerization of alpha-olefin corresponding to the general formula R—CH—CH₂ in which R denotes an alkyl radical, preferably linear alkyl, having from 10 to 50 carbon atoms and preferably from 25 to 50 carbon atoms.

“Homopolymerization of alpha-olefin” means the polymerization of monomers consisting essentially of an alpha-olefin or a mixture of alpha-olefins. These waxes preferably have a number-average molecular weight in the range from 400 to 3000 daltons and in particular from 1800 to 2700 daltons. These polyolefin waxes are described in U.S. Pat. No. 4,060,569 and U.S. Pat. No. 4,239,546. These waxes are notably sold under the name “Performa VR 103”, “Performa VR 253”, “Performa VR 260” by the company New Phase Technology.

(ii) the paraffin waxes having a number-average molecular weight from 350 to 600 daltons, for example the commercial product sold under the name cerafine 56-58 by the company BAERLOCHER.

(iii) the polymethylene waxes that can be obtained by the Fischer-Tropsch process. They generally have a number-average molecular weight in the range from 350 to 600 daltons. In particular, the CIREBELLE waxes manufactured by the company Sasol will be used, such as:

| | | |
|---|---|---|
| CIREBELLE 303: | melting point: | 51° C. |
| CIREBELLE 305: | melting point: | 55° C. |
| CIREBELLE 505: | melting point: | 41° C. |

Among the rheological agents as thickening or gelling agents of the oily phase, as mentioned previously, the following will be used more particularly
(i) semicrystalline polymers, in particular semicrystalline polymers derived from a monomer with a crystallizable chain selected from saturated C₂₄ to C₂₂ alkyl (meth)acrylates and more particularly the poly(stearyl acrylate)s or poly(behenyl acrylate)s.
(ii) lipophilic polyamide polycondensates
(iii) waxes.

Among these thickeners, even more preferably the lipophilic polyamide polycondensates will be used.

### Oily Phase

The oily phase according to the invention generally contains at least one volatile or non-volatile hydrocarbon oil and/or a volatile or non-volatile silicone oil.

“Volatile oil” means, in the sense of the invention, an oil that may evaporate when in contact with the skin or keratin fibres in less than an hour, at room temperature and atmospheric pressure. The volatile oil or oils of the invention are volatile cosmetic oils, liquid at room temperature, having a non-zero vapour pressure, at room temperature and atmospheric pressure, in particular in the range from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

“Non-volatile oil” means an oil that remains on the skin or keratin fibres at room temperature and atmospheric pressure for at least several hours and notably has a vapour pressure below 10⁻³ mmHg (0.13 Pa).

The oily phase preferably represents an amount from 30 to 99.8 wt. % and more preferably from 40 to 90 wt. % relative to the total weight of the composition.

### a) Hydrocarbon Oils

As non-volatile hydrocarbon oils usable according to the invention, we may notably mention:

(i) hydrocarbon oils of vegetable origin such as the triesters of glycerides which are in general triesters of fatty acids and of glycerol, which the fatty acids can have chain lengths from C₄ to C₂₄, the latter being linear or branched, saturated or unsaturated; these oils are notably oils wheatgerm oil, sunflower oil, grapeseed oil, sesame oil, maize oil, apricot oil, castor oil, karite oil, avocado oil, olive oil, soya oil, sweet almond oil, palm oil, colza oil, cotton oil, hazelnut oil, macadamia oil, jojoba oil, alfalfa oil, poppy oil, Chinese okra oil, sesame oil, cucurbit oil, colza oil, blackcurrant oil, evening primrose oil, millet oil, barley oil, quinoa oil, rye oil, safflower oil, candlenut oil, passionflower oil, musk rose oil; or the triglycerides of caprylic/capric acids such as those sold by the company Stéarineries Dubois or those sold under the names Miglyol 810, 812 and 818 by the company Dynamit Nobel,
(ii) synthetic ethers having from 10 to 40 carbon atoms;
(iii) linear or branched hydrocarbons, of mineral or synthetic origin such as petroleum jelly, polydecenes, hydrogenated polyisobutene such as Parleam, squalane, and mixtures thereof;
(iv) synthetic esters such as the oils of formula RCOOR′ in which R represents the residue of a linear or branched fatty acid having from 1 to 40 carbon atoms and R′ represents a hydrocarbon chain, notably branched, containing from 1 to 40 carbon atoms provided that R+R′ is 10, for example purcelline oil (cetostearyl octanoate), isopropyl myristate, isopropyl palmitate, benzoate of C₁₂-C₁₅ alcohols such as the product sold under the trade name “FINSOLV TN” or “WITCONOL TN” by the company WITCO or “TEGOSOFT TN” by the company EVONIK GOLDSCHMIDT, 2-ethylphenyl benzoate such as the commercial product sold under the name “X-TEND 226” by the company ISP, isopropyl lanolate, hexyl laurate, diisopropyl adipate, isononyl isononanoate, oleyl erucate, 2-ethylhexyl palmitate, isostearyl isostearate, octanoates, decanoates or ricinoleates of alcohols or polyalcohols such as propylene glycol dioctanoate; hydroxylated esters such as isostearyl lactate, di-isostearyl malate; and pentaerythritol esters; citrates or tartrates such as of linear C₁₂-C₁₃ di-alkyl tartrates such as those sold under the name COSMACOL ETI by the company ENICHEM AUGUSTA INDUSTRIALE as well as the linear C₁₄-C₁₅ di-alkyl tartrates such as those sold under the name COSMACOL ETL by the same company; the acetates.
(v) fatty alcohols that are liquid at room temperature with a branched and/or unsaturated carbon chain having from 12 to 26 carbon atoms such as octyl dodecanol, isostearyl alcohol, oleic alcohol, 2-hexyldecanol, 2-butyloctanol, 2-undecylpentadecanol;
(vi) higher fatty acids such as oleic acid, linoleic acid, linolenic acid;
(vii) carbonates such as dicaprylyl carbonate such as the product sold under the name “CETIOL CC” by the company COGNIS;
(viii) fatty amides such as isopropyl N-lauroyl sarcosinate such as the product sold under the trade name ELDEW SL205 from AJINOMOTO and mixtures thereof.

The volatile hydrocarbon oils can be selected from the hydrocarbon oils having from 8 to 16 carbon atoms, and notably the branched C₈-C₁₆ alkanes such as the C₈-C₁₆ isoalkanes of petroleum origin (also called isoparaffins) such as isododecane (also called 2,2,4,4,6-pentamethylheptane), isodecane, isohexadecane, alkanes described in the patent applications from the company Cognis WO 2007/068371, or WO2008/155059 (mixtures of various alkanes, differing by at least one carbon). These alkanes are obtained from fatty alcohols, obtained in their turn from copra oil or palm oil, the oils sold under the trade names Isopars or Permethyls, the C₈-C₁₆ branched esters, iso-hexyl neopentanoate, and mixtures thereof. Other volatile hydrocarbon oils such as petroleum distillates, notably those sold under the name Shell Solt by the company SHELL, can also be used. According to one embodiment, the volatile solvent is selected from the volatile hydrocarbon oils having from 8 to 16 carbon atoms and mixtures thereof.

Among the hydrocarbon oils that can be used according to the invention, more particularly the glyceride triesters will be preferred, and notably the triglycerides of caprylic/capric acids, synthetic esters and notably isononyl isononanoate, oleyl erucate, benzoate of C₁₂-C₁₅ alcohols and fatty alcohols notably octyldodecanol.

### b) Silicone Oils

The non-volatile silicone oils can be selected notably from the non-volatile polydimethylsiloxanes (PDMS), the polydimethylsiloxanes having alkyl or alkoxy groups, pendant and/or at the end of the silicone chain, groups each having from 2 to 24 carbon atoms, phenylated silicones such as phenyl trimethicones, phenyl dimethicones, phenyl trimethylsiloxy diphenylsiloxanes, diphenyl dimethicones, diphenyl methyldiphenyl trisiloxanes, 2-phenylethyl trimethylsiloxysilicates.

As volatile silicone oils, we may mention for example the volatile linear or cyclic silicone oils, notably those having a viscosity≦8 centistokes (8 10⁻⁶ m²/s), and notably having from 2 to 7 silicon atoms, said silicones optionally having alkyl or alkoxy groups with from 1 to 10 carbon atoms. As volatile silicone oil usable in the invention, we may notably mention octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

We may also mention the volatile linear alkyltrisiloxane oils of general formula (I):

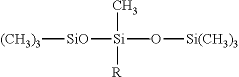

where R represents an alkyl group comprising 2 to 4 carbon atoms and in which one or more hydrogen atoms can be substituted with a fluorine or chlorine atom.

Among the oils of general formula (I), we may mention: 3-butyl-1,1,1,3,5,5,5-heptamethyl trisiloxane, 3-propyl-1,1,1,3,5,5,5-heptamethyl trisiloxane, and 3-ethyl-1,1,1,3,5,5,5-heptamethyl trisiloxane, corresponding to the oils of formula (I) for which R is respectively a butyl group, a propyl group or an ethyl group.

### Linear C₁-C₃ Monohydric Alcohol

According to a particularly preferred embodiment of the invention, the compositions of the invention additionally contain at least one C₁-C₃ monohydric alcohol selected from methanol, ethanol, propanol or mixtures thereof. More particularly, ethanol will be selected.

Said monohydric alcohol(s) are preferably present at concentrations in the range from 0.1 to 40 wt. %, more preferably from 2 to 10 wt. % relative to the total weight of the composition.

### Additional Lipophilic Filters

According to a particularly preferred embodiment of the invention, the compositions of the invention further contain at least one lipophilic organic filter notably selected from derivatives of para-aminobenzoic acid, salicylic derivatives, cinnamic derivatives, benzophenones and aminobenzophenones, anthranilic derivatives, derivatives of dibenzoylmethane, derivatives of β,β-diphenylacrylate, derivatives of benzylidene camphor, derivatives of phenyl benzimidazole, derivatives of benzotriazole, triazine derivatives other than those of formula (I), bis-resorcinyl triazines, derivatives of imidazolines, derivatives of benzalmalonate, derivatives of 4,4-diarylbutadiene, derivatives of benzoxazole, merocyanines and mixtures thereof.

Among the organic lipophilic UVA filters that can absorb UV from 320 to 400 nm, we may mention

### Derivatives of Dibenzoylmethane:

4-isopropyl-dibenzoylmethane, sold under the name “EUSOLEX 8020” by the company MERCK, and corresponding to the following formula:

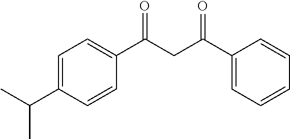

1-(4-methoxy-1-benzofuran-5-yl)-3-phenylpropane-1,3-dione, marketed by the company QUEST under the name Pongamol of formula:

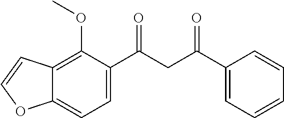

1-(4-tert-butylphenyl)-3-(2-hydroxyphenyl)propane-1,3-dione of formula:

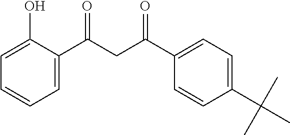

butyl methoxydibenzoylmethane notably sold under the trade name “PARSOL 1789” by HOFFMANN LA ROCHE,

### Aminobenzophenones

n-Hexyl 2-(4-diethylamino-2-hydroxybenzoyl)benzoate sold under the trade name “UVINUL A +”

### Anthranilic Derivatives:

Menthyl anthranilate sold under the trade name “NEO HELIOPAN MA” by HAARMANN et REIMER,

Derivatives of 4,4-diarylbutadiene:

1,1-Dicarboxy (2,2′-dimethylpropyl)-4,4-diphenylbutadiene

Those preferred are:

Butyl methoxydibenzoylmethane
n-Hexyl 2-(4-diethylamino-2-hydroxybenzoyl)benzoate

Among the organic lipophilic UVB filters that can absorb UV from 280 to 320 nm, we may mention

### Para-aminobenzoates:

### Ethyl PABA

### Ethyl Dihydroxypropyl PABA

Ethylhexyl Dimethyl PABA (ESCALOL 507 from ISP)

### Salicylic Derivatives:

Homosalate sold under the name “Eusolex HMS” by Rona/EM Industries,
Ethylhexyl Salicylate sold under the name “NEO HELIOPAN OS” by HAARMANN et REIMER,
Dipropyleneglycol Salicylate sold under the name “DIPSAL” by SCHER,
   TEA Salicylate, sold under the name “NEO HELIOPAN TS” by HAARMANN et REIMER,

### Cinnamates

Ethylhexyl Methoxycinnamate notably sold under the trade name “PARSOL MCX” by HOFFMANN LA ROCHE,
Isopropyl Methoxy cinnamate,
Isoamyl Methoxy cinnamate sold under the trade name “NEO HELIOPAN E 1000” by HAARMANN et REIMER,

### Diisopropyl Methylcinnamate,

### Cinoxate,

### Glyceryl Ethylhexanoate Dimethoxycinnamate

Derivatives of β,β′-diphenylacrylate:
Octocrylene, notably sold under the trade name “UVINUL N539” by BASF,
Etocrylene, notably sold under the trade name “UVINUL N35” by BASF,

### Derivatives of Benzylidene Camphor:

3-Benzylidene camphor manufactured under the name “MEXORYL SD” by CHIMEX,
Methylbenzylidene camphor sold under the name “EUSOLEX 6300” by MERCK,
Polyacrylamidomethyl Benzylidene Camphor manufactured under the name “MEXORYL SW” by CHIMEX,

### Triazine Derivatives:

Ethylhexyl triazone notably sold under the trade name “UVINUL T150” by BASF,
2,4,6-tris(dineopentyl 4′-amino benzalmalonate)-s-triazine
the 2,4,6-tris-(diisobutyl 4′-amino benzalmalonate)-s-triazine,
2,4-bis(dineopentyl 4′-amino benzalmalonate)-6-(n-butyl 4′-aminobenzoate)-s-triazine,
2,4-bis(n-butyl 4′-amino benzoate)-6-(aminopropyltrisiloxane)-s-triazine,

### Derivatives of Imidazolines:

### Ethylhexyl Dimethoxybenzylidene Dioxoimidazoline Propionate,

### Derivatives of Benzalmalonate:

Polyorganosiloxanes with a benzalmalonate function such as Polysilicone-15 sold under the trade name “PARSOL SLX” by HOFFMANN LA ROCHE
Di-neopentyl-4′-methoxybenzalmalonate,

### Derivatives of Merocyanine

Octyl-5-N,N-diethylamino-2-phenysulphonyl-2,4-pentadienoate

Those preferred are:

### Homosalate

### Ethylhexyl Salicylate

### Ethylhexyl Methoxycinnamate

### Octocrylene

### Ethylhexyl Triazone

2,4-bis(n-butyl 4′-amino benzoate)-6-(aminopropyltrisiloxane)-s-triazine,
Octyl-5-N,N-diethylamino-2-phenysulphonyl-2,4-pentadienoate

Among the wide-spectrum organic lipophilic filters that can absorb UVA and UVB, we may mention

### Derivatives of Benzophenone

Benzophenone-1 sold under the trade name “UVINUL 400” by BASF,
Benzophenone-2 sold under the trade name “UVINUL D50” by BASF
Benzophenone-3 or Oxybenzone, sold under the trade name “UVINUL M40” by BASF,

### Benzophenone-5,

Benzophenone-6 sold under the trade name “Helisorb 11” by Norquay
Benzophenone-8 sold under the trade name “Spectra-Sorb UV-24” by American Cyanamid

### Benzophenone-10,

### Benzophenone-11,

### Benzophenone-12,

### Derivatives of Benzotriazole:

Drometrizole Trisiloxane sold under the name “Silatrizole” by RHODIA CHEMISTRY
Bumetrizole sold under the name TINOGUARD AS by CIBA-GEIGY

### The Bis-Resorcinyl Triazine Derivatives

Bis-Ethylhexyloxyphenol Methoxyphenyl Triazine sold under the trade name “TINOSORB S” by CIBA GEIGY,

### Derivatives of Benzoxazole:

2,4-bis-[5-1(dimethylpropyl)benzoxazol-2-yl-(4-phenyl)-imino]-6-(2-ethylhexyl)-imino-1,3,5-triazine sold under the name Uvasorb K2A by Sigma 3V

Those preferred are:

### Benzophenone-3

### Drometrizole Trisiloxane

### Bis-Ethylhexyloxyphenol Methoxyphenyl Triazine

The lipophilic organic filters are generally present in the compositions according to the invention in proportions in the range from 0.1 to 50 wt. % relative to the total weight of the composition, and preferably in the range from 2 to 30 wt. % relative to the total weight of the composition.

According to a particular embodiment of the invention, the compositions will be transparent and will preferably have a turbidity less than 1000 NTU (Nephelometric Turbidity Units) at 25° C., preferably less than 50 NTU at 25° C. and even more preferably less than 15 NTU, measured with a 2100P Turbidimeter from the company HACH. (to be checked)

According to a particularly preferred embodiment of the invention, the compositions will have an SPF greater than 10 or greater than 15 and even greater than 20.

According to a particularly preferred embodiment of the invention, the compositions will have a UVA_{PPD} PF greater than 5, and they are also to meet the regulations notably European that stipulate that the ratio SPF/PPD should be less than 3.

### Additives

The oily composition of the product of the invention can also contain various additives, which can be soluble in the oily phase, or can be dispersed in said oily phase, notably selected from lipophilic dyes, lipophilic active substances, organic solvents, preservatives, insect repellents, essential oils, perfumes, emollients, propellants.

Among the lipophilic cosmetic active substances, we may mention for example antioxidants, keratolytic agents such as N-alkyl salicylic acids, for example N-octanoyl-5-salicylic acid; vitamins such as vitamin E (tocopherol and derivatives), vitamin A (retinol and derivatives); emollients and any lipophilic active substance usually employed in the care of the skin or the hair.

Of course, a person skilled in the art will take care to select any complementary compound or compounds mentioned above and/or the amounts thereof in such a way that the advantageous properties associated intrinsically with the compositions according to the invention are not, or not substantially, impaired by the addition or additions envisaged.

Another object of the present invention comprises the use of the compositions according to the invention as defined above as product for the care cosmetic and/or make-up of the skin, nails, hair, eyelashes, eyebrows and/or of the scalp, notably as care product, and sun protection product.

The cosmetic compositions according to the invention can for example be used as a product with a liquid consistency for care and/or sun protection and/or everyday photoprotection and/or for make-up for the face and/or the body and/or the hair.

### Additional Colourants

According to another particular embodiment of the invention, the compositions of the invention can also include one or more additional colourants.

The additional colourants can also be selected from the synthetic or natural direct dyes. They can be organic or mineral colourants.

The fat-soluble organic dyes, synthetic or natural, are for example DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto, curcumin.

The additional colourants can also be selected from the particulate colourants which are preferably selected from pigments, nacres or interference pigments, glitter.

The term “pigments” means particles of any shape, white or coloured, mineral or organic, insoluble in the physiological medium, intended for colouring the composition.

The pigments can be white or coloured, mineral and/or organic. We may mention, among the mineral pigments, titanium dioxide, optionally surface-treated, oxides of zirconium or of cerium, as well as oxides of zinc, of iron (black, yellow or red) or of chromium, manganese violet, ultramarine, chromium hydrate and ferric blue, metal powders such as aluminium powder, copper powder.

Among the organic pigments, we may mention carbon black, pigments of the D & C type, and lakes based on carmine, barium, strontium, calcium, aluminium.

We may also mention the effect pigments such as particles having an organic or mineral substrate, natural or synthetic, for example glass, acrylic resins, polyester, polyurethane, polyethylene terephthalate, ceramics or aluminas, and said substrate may or may not be covered with metallic substances such as aluminium, gold, silver, platinum, copper, bronze, or with metal oxides such as titanium dioxide, iron oxide, chromium oxide and mixtures thereof.

In the sense of the present invention, the expression “interference particles or nacres” denotes any particle generally possessing a multilayer structure such as to permit the creation of a colour effect by interference of light, which is diffracted and diffused in different ways depending on the nature of the layers. The colouring effects obtained are associated with the lamellar structure of these particles and derive from the physical laws of the optics of thin films (see: Pearl Lustre Pigments—Physical principles, properties, applications—R. Maisch, M. Weigand. Verlag Moderne Industrie). Thus, these particles can give colours that vary depending on the angle of observation and the angle of incidence of the light.

In the sense of the present invention, the term multilayer structure is used in the meaning of a structure formed from a substrate covered with a single layer or a structure formed from a substrate covered with at least two or even several successive layers.

The multilayer structure can thus have one or at least two layers, each layer, independently or not of the other layer(s), being made from at least one material selected from the group comprising the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

Generally, the multilayer structure is of inorganic nature.

More particularly, the interference particles considered according to the invention can be interference pigments, or natural or synthetic nacres, monolayered or multilayered, in particular formed from a natural substrate based inter alia on mica and which is covered with one or more layers of metal oxide.

The interference particles according to the invention are characterized in that 50% of the population by weight has a diameter (d50) less than 40 μm, more particularly less than 30 μm, notably less than 20 μm, and in particular less than 15 μm, measured with a laser granulometer, for example the Mastersizer 2000® from Malvernet or the BI90 +® from Broockhaven Instrument Corporation.

The nacres of the mica/tin oxide/titanium dioxide type are quite particularly suitable for the invention, for example those marketed under the names TIMIRON SILK BLUE®, TIMIRON SILK RED®, TIMIRON SILK GREEN®, TIMIRON SILK GOLD® and TIMIRON SUPER SILK® offered by the company MERCK and the mica/iron oxide/titanium dioxide nacres, for example FLAMENCO SATIN BLUE®, FLAMENCO SATIN RED® and FLAMENCO SATIN VIOLET® and FLAMENCO ORANGE 320C offered by the company ENGELHARD and mixtures thereof.

More precisely, these pigments can be present in amounts in the range from 0.01 to 10 wt. % and preferably in the range from 0.1 to 5 wt. % relative to the total weight of the composition.

### Vaporizable Compositions

The compositions according to the invention can be in the form of vaporizable oil applied on the skin or the hair in the form of fine particles by means of pressurization devices. The devices according to the invention are well known by a person skilled in the art and comprise non-aerosol pumps or “atomizers”, aerosol containers comprising a propellant as well as aerosol pumps using compressed air as propellant. The latter are described in U.S. Pat. No. 4,077,441 and U.S. Pat. No. 4,850,517 (which form an integral part of the contents of the description).

These compositions can also be impregnated on substrates such as wipes, or they can be packaged as lotions in a bottle with a reducer valve.

The aerosol packaged compositions according to the invention generally contain conventional propellants, for example hydrofluorinated compounds, dichlorodifluoromethane, difluoroethane, dimethyl ether, isobutane, n-butane, propane, trichlorofluoromethane. They are preferably present in amounts in the range from 15 to 50 wt. % relative to the total weight of the composition.

Actual examples, but not in any way limiting, illustrating the invention, will now be given.

### EXAMPLE 1

### SPF Vivo=57.3 and UVA_{PPD} PF Vivo=20.9 (5 Subjects)

| | |
|---|---|
| | Amounts |
| Ingredients | wt. % |

Phase A

| | |
|---|---|
| ETHYLENEDIAMINE/STEARYL DIMER | 6.0 |
| DILINOLEATE COPOLYMER (UNICLEAR | |
| 100 VG - ARIZONA CHEMICAL) | |
| CAPRYLIC/CAPRIC TRIGLYCERIDE (60/40) | 33.0 |
| (MYRITOL 318 - COGNIS) | |
| BUTYL METHOXYDIBENZOYLMETHANE | 5.0 |
| ETHYLHEXYL TRIAZONE | 5.0 |
| OCTOCRYLENE | 5.0 |
| ETHYLHEXYL SALICYLATE | 5.0 |
| DROMETRIZOLE TRISILOXANE | 1.0 |
| DIETHYLHEXYL BUTAMIDO TRIAZONE | 3.0 |
| (UVASORB HEB - SIGMA 3V) | |
| C12-15 ALKYL BENZOATE | 13.0 |
| (TEGOSOFT TN from EVONIK GOLDSCHMIDT) | |
| OCTYL-2-DODECANOL | q.s. 100 |

Phase B

| | |
|---|---|
| ETHANOL | 8.0 |
| PERFUME | 0.3 |

The procedure is as follows:

Prepare phase A by mixing the raw materials and heat at 90-95° C. while stirring until all of the raw materials have dissolved. Cool to 25° C. while stirring. Then incorporate phase B at 25° C. while stirring until perfectly homogeneous. Evaluate, for each of the compositions
(1) the SPF in vivo on 5 subjects according to the International method published by Colipa/CTFA SA/JCIA (May 2006);
(2) the UVA_{PPD} PF on 5 subjects according to the recommendations of the JCIA (version dated 15/11/1995).

### EXAMPLES 2 AND 3

Prepare formulations 2 and 3 following the same procedure and evaluate the SPF in vitro for each of the compositions. Measure the mean SPF of each formulation according to the method of evaluation of the protection factor using the in-vitro method described by B. L. DIFFEY et al. in J. Soc. Cosmet. Chem. 40-127-133 (1989), which consists of determining the monochromatic protection factors at 5 nm intervals in a wavelength range from 290 to 400 nm, and calculating, from the values obtained, the sun protection factor according to a given mathematical equation. Each composition tested is applied on plates of quartz+Transpore® with 5 plates per test and 4 measurements per plate. The spectrophotometer used is Labsphere UV 10005.

| | | Ex 3 |
|---|---|---|
| | | (outside |
| | | of the |
| | Ex 2 | invention) |
| Ingredients | wt. % | wt. % |
| ETHYLENEDIAMINE/STEARYL DIMER | 6.0 | — |
| DILINOLEATE COPOLYMER (UNICLEAR 100 | | |
| VG - ARIZONA CHEMICAL) | | |
| CAPRYLIC/CAPRIC TRIGLYCERIDE | 33.0 | 33.0 |
| (60/40) (MYRITOL 318 - COGNIS) | | |
| BUTYL METHOXYDIBENZOYLMETHANE | 3.5 | 3.5 |
| OCTOCRYLENE | 5.0 | 5.0 |
| ETHYLHEXYL SALICYLATE | 5.0 | 5.0 |
| DROMETRIZOLE TRISILOXANE | 1.0 | 1.0 |
| DIETHYLHEXYL BUTAMIDO TRIAZONE | 3.0 | 3.0 |
| C12-15 ALKYL BENZOATE | 13.0 | 13.0 |
| (TEGOSOFT TN from EVONIK | | |
| GOLDSCHMIDT) | | |
| ETHANOL | 3.0 | 3.0 |
| OCTYL-2-DODECANOL | q.s. 100 | q.s. 100 |
| SPF IN VITRO | 100.6 | 36.2 |

These results clearly show that addition of a rheological agent as thickener of the fatty phase leads to a marked increase in SPF.

### EXAMPLES 4 TO 6

Prepare formulations 4 to 6 according to the same procedure and, for each of the compositions, evaluate the flow according to the following protocol:

Carry out the measurements at 25° C. on a Haake RS600 instrument equipped with cone and plate geometry of sanded titanium of 600 mm, degree 2. The measurements are taken in flow with imposed stress, from 0.1 Pa to 100 Pa. The flow is evaluated on the basis of the threshold stresses that correspond to the change of slope on the curves of deformation as a function of stress.

These compositions were constructed based on example 6, replacing some or all of the Ethylhexyl Triazone filter with diethylhexyl butamido triazone, as these two filters have equivalent filtering properties.

| | | | Ex 6 |
|---|---|---|---|
| | | | (outside |
| | | | of the |
| Ingredients | Ex 4 | Ex 5 | invention) |
| ETHYLENEDIAMINE/STEARYL | 6.0 | 6.0 | 6.0 |
| DIMER DILINOLEATE | | | |
| COPOLYMER (UNICLEAR 100 | | | |
| VG - ARIZONA CHEMICAL) | | | |
| CAPRYLIC/CAPRIC | 33.0 | 33.0 | 33.0 |
| TRIGLYCERIDE (60/40) | | | |
| (MYRITOL 318 - COGNIS) | | | |
| BUTYL | 3.5 | 3.5 | 3.5 |
| METHOXYDIBENZOYL- | | | |
| METHANE | | | |
| OCTOCRYLENE | 5.0 | 5.0 | 5.0 |
| ETHYLHEXYL SALICYLATE | 5.0 | 5.0 | 5.0 |
| DROMETRIZOLE TRISILOXANE | 1.0 | 1.0 | 1.0 |
| ETHYLHEXYL TRIAZONE | — | 3.0 | 6.0 |
| DIETHYLHEXYL BUTAMIDO | 6.0 | 3.0 | — |
| TRIAZONE | | | |
| C12-15 ALKYL BENZOATE | 13.0 | 13.0 | 13.0 |
| (TEGOSOFT TN from EVONIK | | | |
| GOLDSCHMIDT) | | | |
| ETHANOL | 5.0 | 5.0 | 5.0 |
| OCTYL-2-DODECANOL | q.s. 100 | q.s. 100 | q.s. 100 |
| Observation of flow | Good flow | Moderate | Poor flow |
| | | flow | |

These results clearly show while maintaining the filtering power of the composition (replacement of Ethylhexyl Triazone with Diethylhexyl Butamido Triazone), the use of Diethylhexyl Butamido Triazone instead of another triazine filter improved the utility properties of the composition, notably its ability to flow.

Other examples according to the invention performed with the procedure given below employing different oil gelling rheological agents are given below for illustrating the invention:

| Ingredients | Ex 7 | Ex 8 | Ex 9 |
|---|---|---|---|
| ETHYLENEDIAMINE/HYDROGENATED | 6.0 | — | — |
| DIMER DILINOLEATE | | | |
| COPOLYMER BIS-DI-C14-18 | | | |
| ALKYL AMIDE (UNICLEAR | | | |
| A200V - ARIZONA | | | |
| CHEMICAL) | | | |
| SYNTHETIC WAX | — | 6.0 | — |
| (CIREBELLE 303 - SASOL) | | | |
| POLY C10-30 ALKYL | — | — | 5.0 |
| ACRYLATE (INTAL IPA 13- | | | |
| 6-AIR PRODUCTS) | | | |
| CAPRYLIC/CAPRIC | 33.0 | 33.0 | 33.0 |
| TRIGLYCERIDE (60/40) | | | |
| (MYRITOL 318 - COGNIS) | | | |
| BUTYL | 3.5 | 3.5 | 3.5 |
| METHOXYDIBENZOYLMETHANE | | | |
| OCTOCRYLENE | 5.0 | 5.0 | 5.0 |
| ETHYLHEXYL SALICYLATE | 5.0 | 5.0 | 5.0 |
| DROMETRIZOLE | 1.0 | 1.0 | 1.0 |
| TRISILOXANE | | | |
| DIETHYLHEXYL BUTAMIDO | 5.0 | 5.0 | 5.0 |
| TRIAZONE | | | |
| C12-15 ALKYL BENZOATE | 13.0 | 13.0 | 13.0 |
| (TEGOSOFT TN from | | | |
| EVONIK GOLDSCHMIDT) | | | |
| ETHANOL | 5.0 | 5.0 | 5.0 |
| OCTYL-2-DODECANOL | q.s. 100 | q.s. 100 | q.s. 100 |

1. Anhydrous fluid composition comprising, in a cosmetically acceptable medium:
a) at least one oily phase and
b) at least one triazine UV filter of the following formula (I): 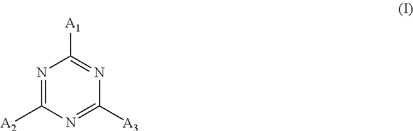
in which the radicals A₁, A₂ and A₃, which may be identical or different, denote a group with the following formula (II): 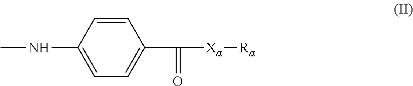
one or two of the groups Xₐ—Rₐ represent the radical —NH—Rₐ with Rₐ selected from: a linear or branched C₁-C₁₈ alkyl radical; a C₅-C₁₂ cycloalkyl radical optionally substituted with one or more C₁-C₄ alkyl radicals; a radical of the following formula (α), (β) or (ε): 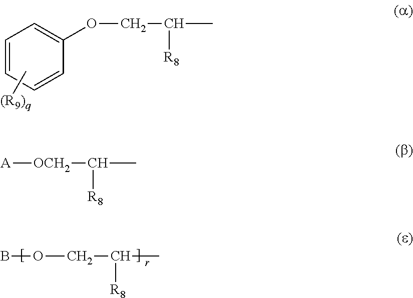
in which:
R₈ is hydrogen or a C₁-C₄ alkyl radical;
R₉ is C₁-C₄ alkyl;
q is an integer from 0 to 3;
r is an integer from 1 to 10;
A is a C₄-C₈ alkyl radical or a C₅-C₈ cycloalkyl radical;
B is a C₁-C₄ alkyl radical;
the other two Xₐ—Rₐ being the radical —O—Rₐ with Rₐ, which may be identical or different, selected from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (α), (β) or (ε) as defined above;
c) at least one rheological agent for thickening or gelling the oily phase.

2. Composition according to claim 1, characterized in that wherein the compound of formula (I) is 2-[(p-(tert-butylamido)anilino]-4,6-bis-[(p-(2′-ethylhexyl-1′-oxycarbonyl)anilino]-1,3,5-triazine or “Diethylhexyl Butamido Triazone” corresponding to the following formula: 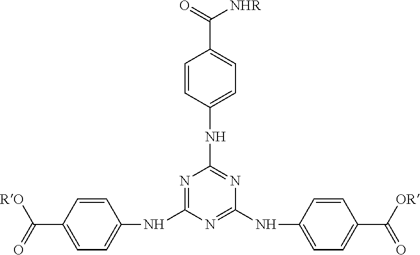
in which R′ denotes an ethyl-2-hexyl radical and R denotes a tert-butyl radical.

3. Composition according to claim 1, wherein the triazine compound of formula (I) is present in proportions in the range from 0.1 to 10 wt. % relative to the total weight of the composition.

4. Composition according to claim 1, wherein the rheological agent is present in amounts in the range from 0.1% to 10% relative to the total weight of the composition.

5. Composition according to claim 1, wherein the rheological agent is selected from:
(1) crystalline polymers,
(2) esters of dextrin and of fatty acid,
(3) modified hydrophobic polysaccharides,
(4) crystalline olefin copolymers
(5) crystalline polycondensates;
(6) lipophilic mineral structure-forming agents,
(7) polymers of the lipophilic polyamide type,
(8) lipophilic polyureas and polyurethanes,
(9) silicone polymers comprising if necessary at least one hydrocarbon unit having two groups capable of establishing hydrogen interactions selected from ester, amide, sulphonamide, carbamate, thiocarbamate, urea, urethane, thiourea, oxamido, guanidino, biguanidino groups and combinations thereof,
(10) organic gelling agents;
(11) block copolymers;
(12) silicone elastomers;
(13) cholesteric liquid crystal agents;
(14) waxes;
and mixtures thereof.

6. Composition according to claim 5, wherein the rheological agent for thickening or gelling the oily phase is selected from:
(i) semicrystalline polymers
(ii) lipophilic polyamide polycondensates
(iii) waxes.

7. Composition according to claim 6, wherein the rheological agent for thickening or gelling the oily phase is a lipophilic polyamide polycondensate.

8. Composition according to claim 1, wherein the oily phase represents an amount from 30 to 99.8 wt. % relative to the total weight of the composition.

9. Composition according to claim 1, further comprising at least one C₁-C₃ monohydric alcohol.

10. Composition according to claim 1, further comprising at least one additional lipophilic organic filter.

11. Composition according to claim 1, wherein the lipophilic organic filter(s) are selected from derivatives of para-aminobenzoic acid, salicylic derivatives, cinnamic derivatives, benzophenones and aminobenzophenones, anthranilic derivatives, of derivatives dibenzoylmethane, derivatives of β,β-diphenylacrylate, derivatives of benzylidene camphor, derivatives of phenyl benzimidazole, derivatives of benzotriazole, triazine derivatives other than those of formula (I), bis-resorcinyl triazines, derivatives of imidazolines, derivatives of benzalmalonate, derivatives of 4,4-diarylbutadiene, derivatives of benzoxazole, merocyanines and mixtures thereof.

12. Composition according to claim 1, wherein it is transparent and has a turbidity less than 1000 NTU at 25° C.

13. Composition according to claim 1, wherein it has an SPF greater than 10.

14. Composition according to claim 1, wherein it has a UVA_{PPD} PF greater than 5 and/or an SPF/PPD ratio of less than 3.

15. A product for cosmetic care and/or make-up and/or for the skin, nails, hair, eyelashes, eyebrows and/or scalp comprising a composition as defined in claim 1.

16. A composition according to claim 1 wherein R₈ and R₉ are each methyl.

17. A composition according to claim 1, wherein the triazine compound of formula (I) is present in proportions in the range from 1 to 6 wt. % relative to the total weight of the composition.

18. A composition according to claim 1, wherein the rheological agent is present in amounts in the range from 1% to 7% relative to the total weight of the composition.

19. A composition according to claim 1, wherein the rheological agent is present in amounts in the range from 4% to 7% relative to the total weight of the composition.

20. A composition according to claim 5, wherein the (1) crystalline polymers are semicrystalline polymers and the (6) lipophilic mineral structure-forming agents are lipophilic clays or hydrophobic silicas and the (9) silicone polymers comprising if necessary amide groups.
